(19) United States
(12) Patent Application Publication
(10) Pub. No.: US 2023/0270967 A1
(43) Pub. Date: Aug. 31, 2023

(54) CONNECTED OXYGEN THERAPY SYSTEM FOR CHRONIC RESPIRATORY DISEASE MANAGEMENT

(71) Applicant: ResMed Asia Pte. Ltd., Singapore (SG)

(72) Inventors: Wai Loon OOI, Singapore (SG); Tirza SUMITRO, Singapore (SG); Teck Wei (Chen Diwei) TAN, Singapore (SG); Khian Boon LIM, Singapore (SG); Jason TJIA, Singapore (SG); Kean Wah LOW, Singapore (SG); Shin Chin LEE, Singapore (SG); Yu Fan LOH, Singapore (SG)

(21) Appl. No.: 18/007,294

(22) PCT Filed: Jul. 27, 2021
(86) PCT No.: PCT/IB2021/056818

§ 371 Date: Jan. 27, 2023

Related U.S. Application Data
(60) Provisional application No. 63/057,607, filed on Jul. 28, 2020.

(51) Int. Cl. A61M 16/10; A61M 16/00; G16H 50/70; G16H 10/60; G16H 20/40; G01N 33/00

(52) U.S. Cl. CPC A61M 16/101; A61M 16/0063; A61M 16/024; G16H 50/70; G16H 10/60; G16H 20/40; G01N 33/0036; A61M 2202/0208; G01N 33/4925

(57) ABSTRACT

A method and system of responding to adverse environmental conditions local to a user of an oxygen concentrator is disclosed. Physiological data of the user is collected. Operational data of the oxygen concentrator is collected during operation of the oxygen concentrator. Environment data local to the oxygen concentrator is collected. Based on the collected environmental data, it is determined whether adverse environmental conditions exist local to the oxygen concentrator. The collected physiological, operational, and environmental data are analyzed to determine a responsive action to the determined adverse environmental conditions. The responsive action is communicated to the user.

### CROSS-REFERENCE TO RELATED APPLICATIONS

This application claims benefit of, and priority to, U.S. Provisional Patent Application No. 63/057,607, filed Jul. 28, 2020, which is hereby incorporated by reference herein in its entirety.

### TECHNICAL FIELD

The present disclosure relates generally to portable oxygen concentrators (POCs), and more specifically to methods and systems for adapting operation of the POC and/or behaviour of a patient using a POC to lessen the effect of poor local air quality.

### BACKGROUND

There are many users (patients) that require supplemental oxygen as part of Long Term Oxygen Therapy (LTOT). Currently, the vast majority of users that are receiving LTOT are diagnosed under the general category of Chronic Obstructive Pulmonary Disease (COPD). This general diagnosis includes common diseases such as Chronic Bronchitis, Emphysema, and related pulmonary conditions. Other users may also require supplemental oxygen, for example, obese individuals to maintain elevated activity levels, users with cystic fibrosis, or infants with broncho-pulmonary dysplasia.

Doctors may prescribe oxygen concentrators or portable tanks of medical oxygen for these users. Usually a specific continuous oxygen flow rate is prescribed (e.g., 1 litre per minute (LPM), 2 LPM, 3 LPM, etc.). Experts in this field have also recognized that exercise for these users provide long term benefits that slow the progression of the disease, improve quality of life and extend user longevity. Most stationary forms of exercise like treadmills and stationary bicycles, however, are too strenuous for these users. As a result, the need for mobility has long been recognized. Until recently, this mobility has been facilitated by the use of small compressed oxygen tanks or cylinders mounted on a cart with dolly wheels. The disadvantage of these tanks is that they contain a finite amount of oxygen and are heavy, weighing about 50 pounds when mounted.

Oxygen concentrators have been in use for about 50 years to supply oxygen for respiratory therapy. Oxygen concentrators may implement cyclic processes such as vacuum swing adsorption (VSA), pressure swing adsorption (PSA), or vacuum pressure swing adsorption (VPSA). For example, oxygen concentrators, e.g., POCs, may work based on depressurization (e.g., vacuum operation) and/or pressurization (e.g., compressor operation) in a swing adsorption process (e.g., Vacuum Swing Adsorption, Pressure Swing Adsorption or Vacuum Pressure Swing Adsorption, each of which are referred to herein as a “swing adsorption process”). Pressure swing adsorption may involve using one or more compressors to increase gas pressure inside one or more canisters that contains particles of a gas separation adsorbent. Such a canister containing a mass of gas separation adsorbent, such as a layer of gas separation adsorbent, may serve as a sieve bed. As the pressure increases, certain molecules in the gas may become adsorbed onto the gas separation adsorbent. Removal of a portion of the gas in the canister under the pressurized conditions allows separation of the non-adsorbed molecules from the adsorbed molecules. The adsorbed molecules may then be desorbed by venting the sieve beds. Further details regarding oxygen concentrators may be found, for example, in U.S. Published Patent Application No. 2009-0065007, published Mar. 12, 2009, and entitled “Oxygen Concentrator Apparatus and Method”, which is incorporated herein by reference.

Ambient air usually includes approximately 78% nitrogen and 21% oxygen with the balance comprised of argon, carbon dioxide, water vapor and other trace gases. If a gas mixture such as air, for example, is passed under pressure through a canister containing a gas separation adsorbent that attracts nitrogen more strongly than it does oxygen, part or all of the nitrogen will stay in the canister, and the gas coming out of the canister will be enriched in oxygen. When the sieve bed reaches the end of its capacity to adsorb nitrogen, the adsorbed nitrogen may be desorbed by venting. The sieve bed is then ready for another cycle of producing oxygen enriched air. By alternating pressurization of the canisters in a two-canister system, one canister can be separating oxygen while the other canister is being vented (resulting in a near-continuous separation of oxygen from the air). In this manner, oxygen enriched air can be accumulated, such as in a storage container or other pressurizable vessel or conduit coupled to the canisters, for a variety of uses including providing supplemental oxygen to users.

Vacuum swing adsorption (VSA) provides an alternative gas separation technique. VSA typically draws the gas through the separation process of the sieve beds using a vacuum such as a compressor configured to create a vacuum within the sieve beds. Vacuum Pressure Swing Adsorption (VPSA) may be understood to be a hybrid system using a combined vacuum and pressurization technique. For example, a VPSA system may pressurize the sieve beds for the separation process and also apply a vacuum for depressurizing the sieve beds.

Traditional oxygen concentrators have been bulky and heavy making ordinary ambulatory activities with them difficult and impractical. Recently, companies that manufacture large stationary oxygen concentrators began developing portable oxygen concentrators (POCs). The advantage of POCs is that they can produce a theoretically endless supply of oxygen and provide mobility for patients. In order to make these devices small for mobility, the various systems necessary for the production of oxygen enriched air are condensed. POCs seek to utilize their produced oxygen as efficiently as possible, in order to minimize weight, size, and power consumption. In some implementations, this may be achieved by delivering the oxygen as series of pulses, each pulse or “bolus” timed to coincide with the onset of inhalation. This therapy mode is known as pulsed oxygen delivery (POD) or demand mode, in contrast with traditional continuous flow delivery more suited to stationary oxygen concentrators. POD mode may be implemented with a conserver, which is essentially an active valve with a sensor to determine the onset of inhalation.

Poor local air quality, as measured for example by air quality index (AQI) or pollen count, can both affect the operation of a POC and worsen an oxygen patient's condition. For example, short-term exposure to PM2.5 (inhalable particles of diameter 2.5 micrometers or smaller) above a certain threshold of density might result in acute declines of blood oxygen saturation level (SpO₂) within minutes, and the effects would last for several hours. As another example, a POC may contain one or more particulate filters to remove particulates above a certain size from the gas flow path. If such a filter becomes clogged by removed particles before its expected lifetime, which may happen if air quality is unexpectedly poor, the POC may have to work harder to deliver the prescribed oxygen dosage, affecting component and battery life. Alternatively, particles that escape the filters could lodge elsewhere in the gas path, such as in the valves, and have a similar effect.

A need therefore exists for oxygen therapy systems capable of adapting the operation of the POC and/or the behaviour of an oxygen patient to lessen the effect of poor air quality in the environment of the patient.

### SUMMARY

The present disclosure relates to a connected oxygen therapy system for chronic respiratory disease management that is responsive to local environmental conditions, in particular air quality. As part of the system, a POC sends operational data, e.g. usage, output impedance, patient parameters such as breathing rate, and geolocation to a remote server. The server correlates the geolocation with a public environmental database or other information source to obtain environmental conditions, e.g. air quality, local to the POC. Alternatively, the POC could contain or communicate with a particulate sensor to measure and send local air quality data to the server. The server analyses the data and takes or recommends action to lessen the effects of adverse local environmental conditions, such as poor air quality, on the patient using the POC.

One disclosed example method is responding to adverse environmental conditions local to a user of an oxygen concentrator is disclosed. Physiological data of the user is collected. Operational data of the oxygen concentrator is collected during operation of the oxygen concentrator. Environment data local to the oxygen concentrator is collected. Based on the collected environmental data, it is determined whether adverse environmental conditions exist local to the oxygen concentrator. The collected physiological, operational, and environmental data are analyzed to determine a responsive action to the determined adverse environmental conditions. The responsive action is communicated to the user.

A further implementation of the example method is where the operational data comprises geolocation data. A further implementation is where collecting the environmental data comprises retrieving environmental data from an environmental database using the geolocation data. A further implementation is where the environmental data comprises an air quality measure. A further implementation is where determining whether adverse environmental conditions exist comprises comparing the air quality measure to a threshold representative of poor air quality. A further implementation is where the analysing uses physiological, operational, and environmental data collected from other portable oxygen concentrators stored in a database. A further implementation is where the method includes storing the collected physiological, operational, and environmental data in the database. A further implementation is where the responsive action is controlling the oxygen concentrator to change flow control of oxygen to the user.

Another disclosed example is a connected oxygen therapy system including an oxygen concentrator configured to generate oxygen enriched air for delivery to a user. A physiological sensor configured to collect physiological data of the user. An operational sensor is configured to collect operational data of the oxygen concentrator during operation of the oxygen concentrator. The system includes a processor that collects physiological data of the user and collects operational data of the oxygen concentrator during operation of the oxygen concentrator. The processor collects environment data local to the oxygen concentrator. Based on the collected environmental data, the processor determines whether adverse environmental conditions exist local to the oxygen concentrator. The collected physiological, operational, and environmental data are analyzed by the processor to determine a responsive action to the determined adverse environmental conditions. The processor communicates the responsive action to the user.

A further implementation of the example system includes a server in communication with the oxygen concentrator. The processor is a processor of the server. A further implementation of the system includes a portable computing device configured to act as an intermediary between the oxygen concentrator and the server. A further implementation of the system includes an environmental sensor configured to generate the environmental data local to the oxygen concentrator. A further implementation of the system includes a geolocation device configured to generate geolocation data of the oxygen concentrator. A further implementation of the system includes an environmental database comprising environmental data. A further implementation of the system is where the processor is configured to retrieve the environmental data from the environmental database using the geolocation data. A further implementation of the system includes a database on which physiological, operational, and environmental data from other oxygen concentrators are stored. A further implementation of the system is where the processor is configured to use the physiological, operational, and environmental data from other oxygen concentrators in the analysing. A further implementation of the system is where the responsive action is controlling the oxygen concentrator to change flow control of oxygen to the user. The processor is configured to control the oxygen concentrator.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The disclosure will be better understood from the following description of exemplary embodiments together with reference to the accompanying drawings, in which:

FIG. 1A depicts an oxygen concentrator in accordance with one form of the present technology;

FIG. 1B is a schematic diagram of the components of the oxygen concentrator of FIG. 1A;

FIG. 1C is a side view of the main components of the oxygen concentrator of FIG. 1A;

FIG. 1D is a perspective side view of a compression system of the oxygen concentrator of FIG. 1A;

FIG. 1E is a side view of a compression system that includes a heat exchange conduit;

FIG. 1F is a schematic diagram of example outlet components of the oxygen concentrator of FIG. 1A;

FIG. 1G depicts an outlet conduit for the oxygen concentrator of FIG. 1A;

FIG. 1H depicts an alternate outlet conduit for the oxygen concentrator of FIG. 1A;

FIG. 1I is a perspective view of a disassembled canister system for the oxygen concentrator of FIG. 1A;

FIG. 1J is an end view of the canister system of FIG. 1I;

FIG. 1K is an assembled view of the canister system end depicted in FIG. 1J;

FIG. 1L is a view of an opposing end of the canister system of FIG. 1I to that depicted in FIGS. 1J and 1K;

FIG. 1M is an assembled view of the canister system end depicted in FIG. 1L;

FIG. 1N is a block diagram of a communication arrangement of example devices that may be in communication with the oxygen concentrator of FIG. 1A;

FIG. 1O depicts an example control panel for the oxygen concentrator of FIG. 1A;

FIG. 2 is a block diagram of a connected oxygen therapy system that allows data collection from POCs and analysis of that data;

FIG. 3 is a flow chart illustrating a method of responding to local environmental conditions, as implemented in a connected oxygen therapy system such as the system of FIG. 2.

The present disclosure is susceptible to various modifications and alternative forms. Some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION OF THE ILLUSTRATED EMBODIMENTS

The present inventions can be embodied in many different forms. Representative embodiments are shown in the drawings, and will herein be described in detail. The present disclosure is an example or illustration of the principles of the present disclosure, and is not intended to limit the broad aspects of the disclosure to the embodiments illustrated. To that extent, elements and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed, the singular includes the plural and vice versa; and the word “including” means “including without limitation.” Moreover, words of approximation, such as “about,” “almost,” “substantially,” “approximately,” and the like, can be used herein to mean “at,” “near,” or “nearly at,” or “within 3-5% of,” or “within acceptable manufacturing tolerances,” or any logical combination thereof, for example.

The present disclosure relates to a system that leverages operational data collected by multiple oxygen concentrator devices to provide analysis of patient health conditions in a cloud-based engine. The system provides a connected-care service value in the health care (and particularly Integrated Care) market, thereby reducing the burden of care by incorporating sensing technology in oxygen concentrator devices and services, supported by communications and machine-learning technologies.

An example oxygen concentrator device such as a portable oxygen concentrator (POC) may monitor and collect operational data, such as geolocation and therapy usage, and physiological data such as breathing rate and inspiratory time. The example POC may also collect physiological data from additional sensors on a connected body patch or health monitoring device with electrical or optical sensing, such as a smart watch, bracelet, or ring. The example POC may also collect local environmental data from additional sensors in communication with the POC. Data could also be integrated from other therapy devices, such as a smart inhaler, used by the patient. The example POC may transmit the collected data to a remote computing device such as a server over a network.

The present disclosure also relates to a health data analysis engine running on the server. The health data analysis engine is configured to collect the data transmitted by the oxygen concentrator, analyse the data, and take or recommend action to lessen the effects of adverse local environmental conditions such as poor air quality on the patient using the POC.

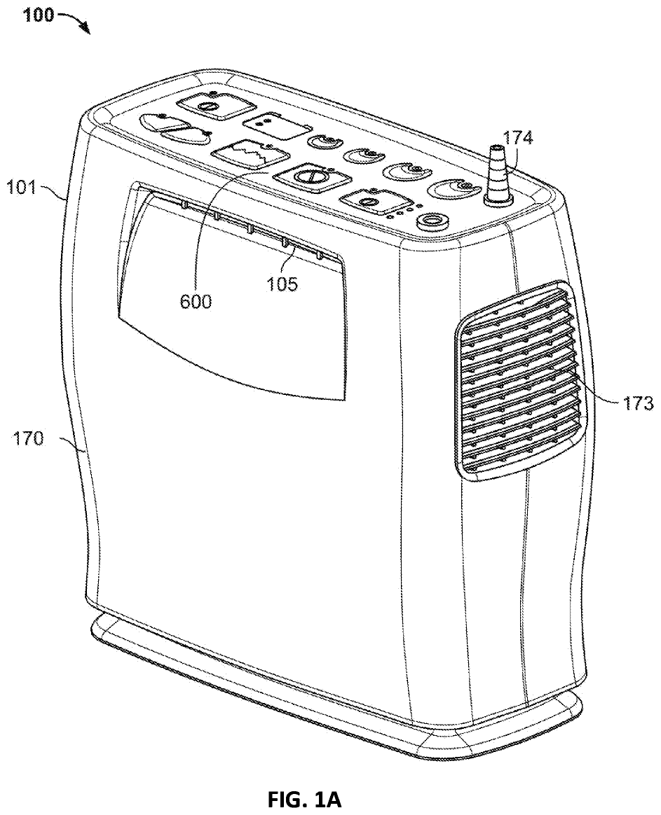

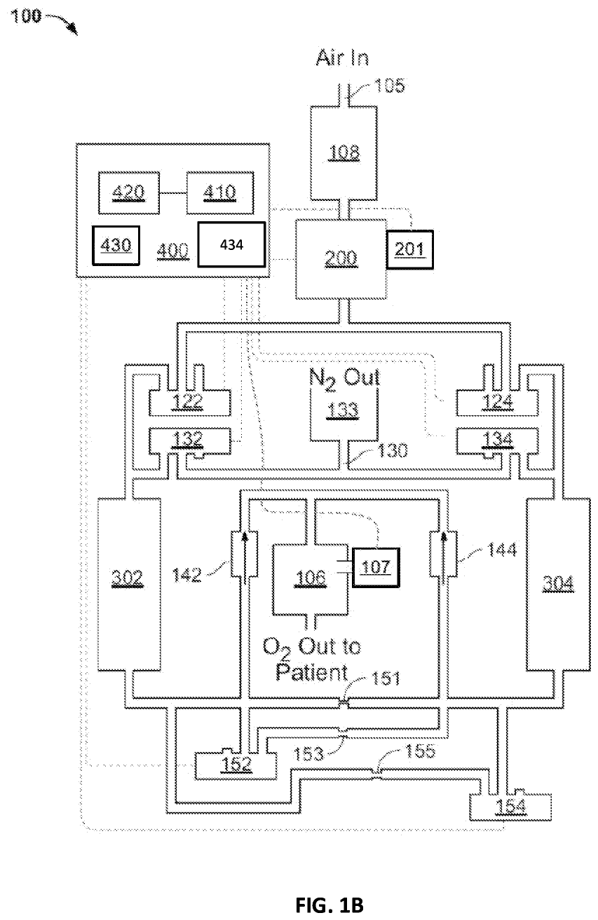

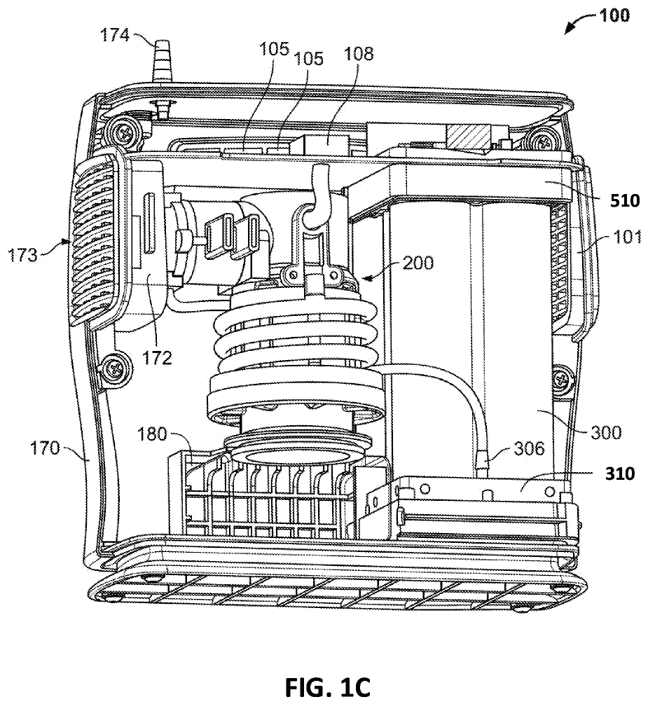

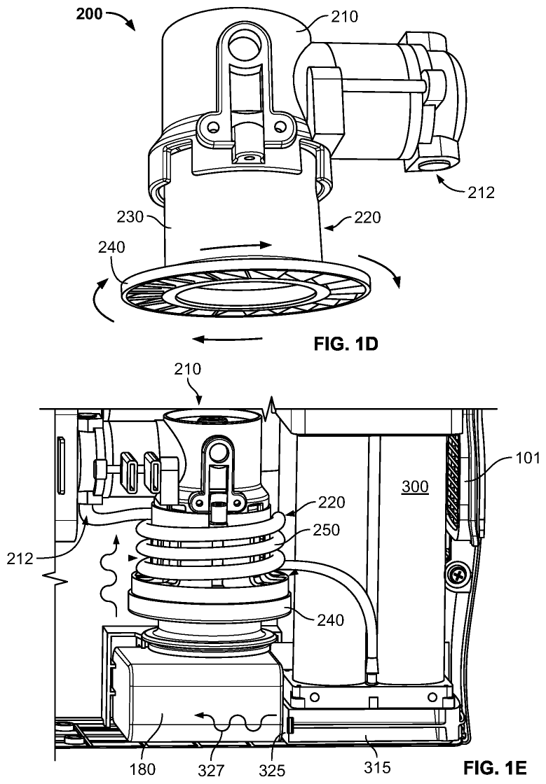

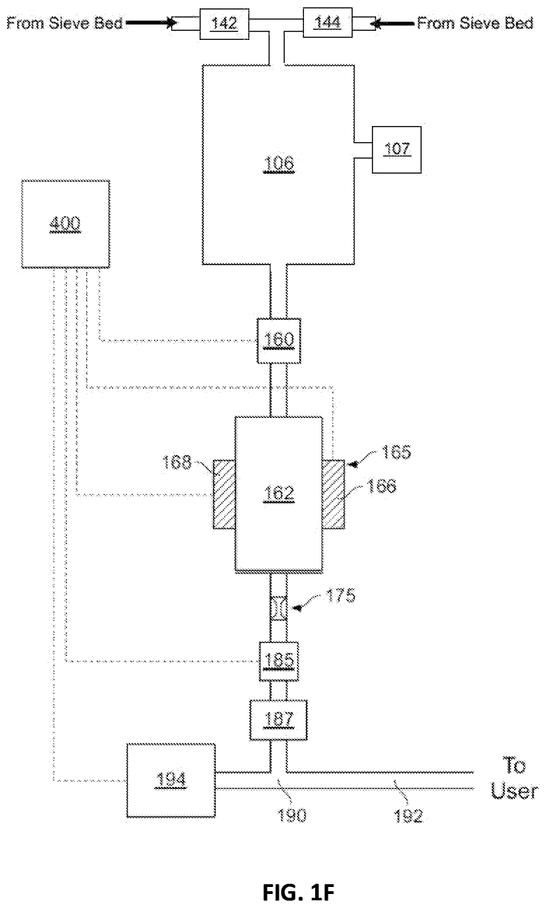

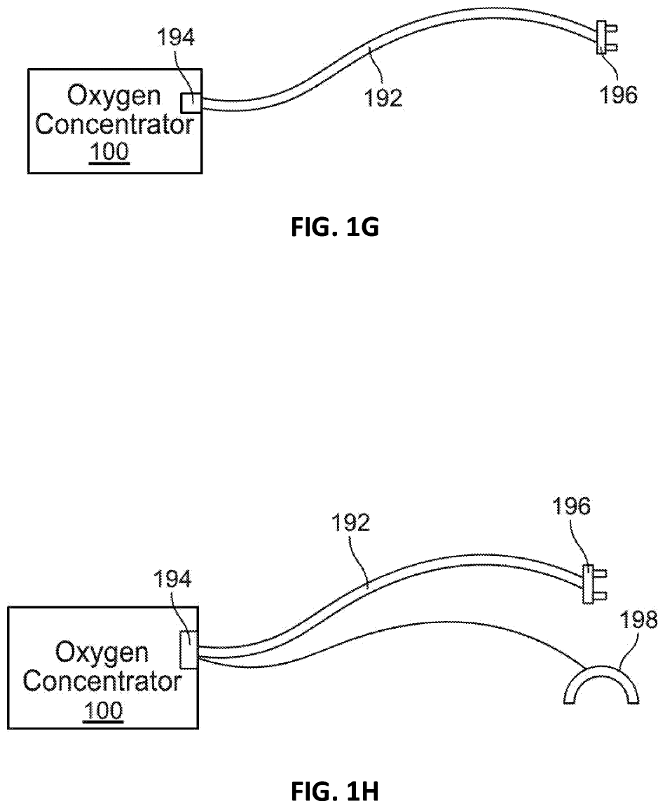

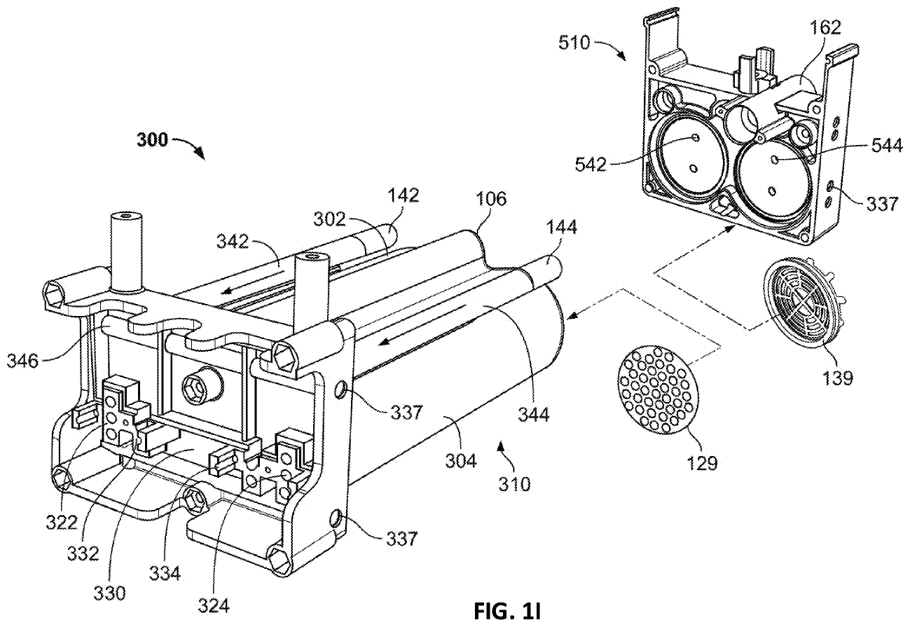

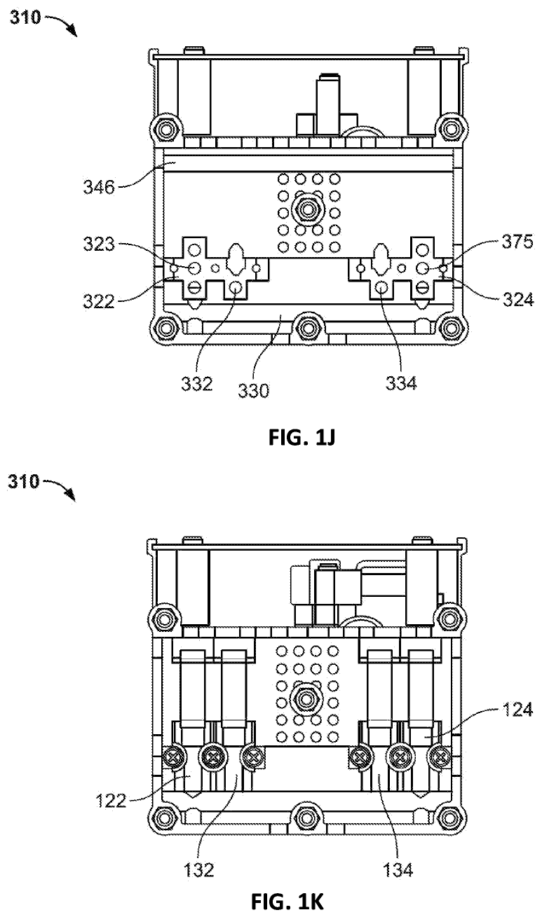

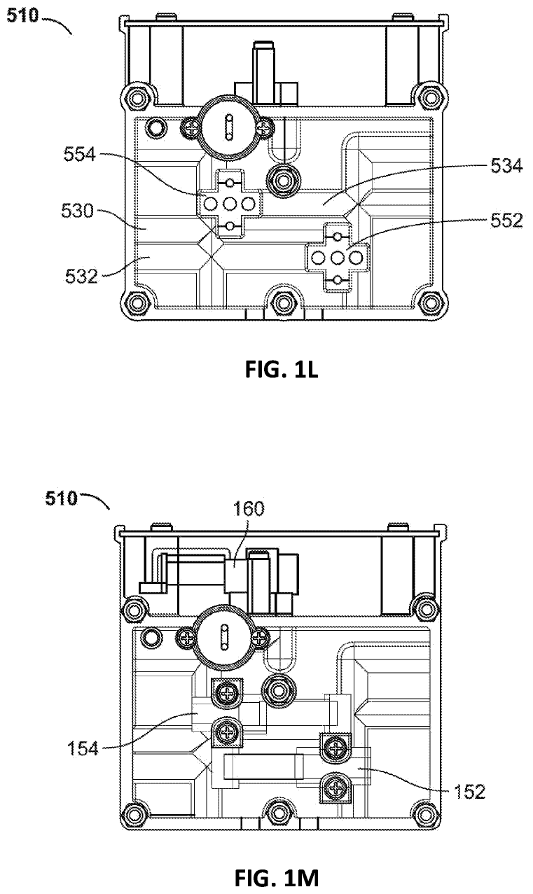

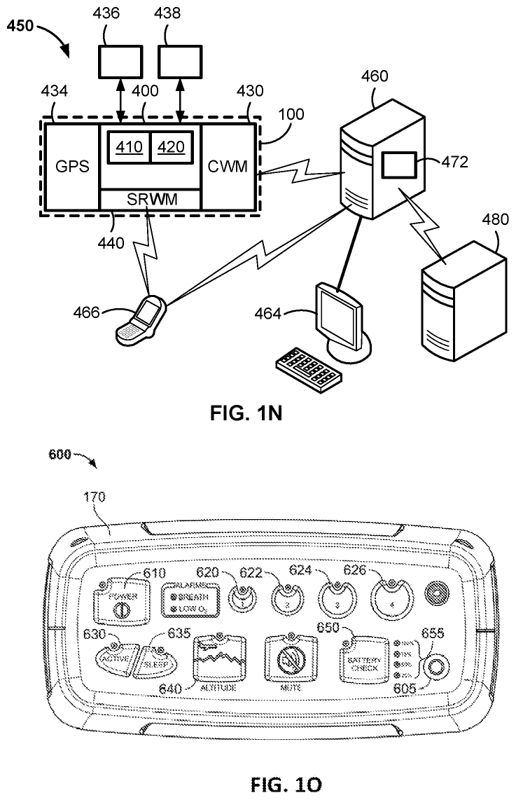

FIGS. 1A-1N illustrate an implementation of an oxygen concentrator 100. As described herein, the oxygen concentrator 100 uses a cyclic pressure swing adsorption (PSA) process to produce oxygen enriched air. However, in other embodiments, oxygen concentrator 100 may be modified such that it uses a cyclic vacuum swing adsorption (VSA) process or a cyclic vacuum pressure swing adsorption (VPSA) process to produce oxygen enriched air. The examples of the present technology may be implemented with any of the following structures and operations.

FIG. 1A depicts an implementation of an outer housing 170 of an oxygen concentrator 100. In some implementations, outer housing 170 may be comprised of a light-weight plastic. The outer housing 170 includes compression system inlets 105, cooling system passive inlet 101 and outlet 173 at each end of the outer housing 170, outlet port 174, and control panel 600. Inlet 101 and outlet 173 allow cooling air to enter the housing, flow through the housing, and exit the interior of housing 170 to aid in cooling of the oxygen concentrator 100. The compression system inlets 105 allow air to enter the compression system. The outlet port 174 is used to attach a conduit to provide oxygen enriched air produced by the oxygen concentrator 100 to a user.

FIG. 1B illustrates a schematic diagram of components of the example oxygen concentrator 100 in FIG. 1A. Oxygen concentrator 100 may concentrate oxygen within an air stream to provide oxygen enriched air to a user. As used herein, “oxygen enriched air” is a gas mixture composed of at least about 50% oxygen, at least about 60% oxygen, at least about 70% oxygen, at least about 80% oxygen, at least about 90% oxygen, at least about 95% oxygen, at least about 98% oxygen, or at least about 99% oxygen. One example of a minimal range is 86-87% oxygen for portable oxygen concentrators.

Oxygen concentrator 100 may be a portable oxygen concentrator. For example, the oxygen concentrator 100 may have a weight and size that allows the oxygen concentrator 100 to be carried by hand and/or in a carrying case. In one implementation, oxygen concentrator 100 has a weight of less than about 20 pounds (9.7 kg), less than about 15 pounds (6.80 kg), less than about 10 pounds (4.54 kg), or less than about 5 pounds (2.27 kg). In an implementation, the oxygen concentrator 100 has a volume of less than about 1000 cubic inches (0.0164 cubic meters), less than about 750 cubic inches (0.0123 cubic meters); less than about 500 cubic inches (0.0082 cubic meters), less than about 250 cubic inches (0.0041 cubic meters), or less than about 200 cubic inches (0.0033 cubic meters).

Oxygen enriched air may be produced from ambient air by pressurizing ambient air in a sieve bed in the form of canisters 302 and 304, which include a gas separation adsorbent. Gas separation adsorbents useful in an oxygen concentrator are capable of separating at least nitrogen from an air stream to produce oxygen enriched air. Examples of gas separation adsorbents include molecular sieves that are capable of separating nitrogen from an air stream. Examples of adsorbents that may be used in an oxygen concentrator include, but are not limited to, zeolites (natural) or synthetic crystalline aluminosilicates that separate nitrogen from an air stream under elevated pressure. Examples of synthetic crystalline aluminosilicates that may be used include, but are not limited to: OXYSIV adsorbents available from UOP LLC, Des Plaines, Ill.; SYLOBEAD adsorbents available from W. R. Grace & Co, Columbia, Md.; SILIPORITE adsorbents available from CECA S.A. of Paris, France; ZEOCHEM adsorbents available from Zeochem AG, Uetikon, Switzerland; and AgLiLSX adsorbent available from Air Products and Chemicals, Inc., Allentown, Pa.

As shown in FIG. 1B, air may enter the oxygen concentrator 100 through air inlet 105. Air may be drawn into air inlet 105 by compression system 200. Compression system 200 may draw in air from the surroundings of the oxygen concentrator and compress the air, forcing the compressed air into one or both canisters 302 and 304. In an implementation, an inlet muffler 108 may be coupled to air inlet 105 to reduce sound produced by air being pulled into the oxygen concentrator by compression system 200. In an implementation, inlet muffler 108 may be used to reduce moisture and sound. For example, a moisture adsorbent material (such as a polymer water adsorbent material or a zeolite material) may be used to both adsorb moisture, i.e. water, from the incoming air and to reduce the sound of the air passing into the air inlet 105.

Compression system 200 may include one or more compressors configured to compress air. Pressurized air, produced by compression system 200, may be fed into one or both of the canisters 302 and 304. In some implementations, the ambient air may be pressurized in the canisters to a pressure approximately in a range of 13-20 pounds per square inch (psi) (89.6-137.9 kPa) gauge pressure (psig). Other pressures may also be used, depending on the type of gas separation adsorbent disposed in the canisters.

Coupled to each canister 302 and 304 are inlet valves 122 and 124 and outlet valves 132 and 134. As shown in FIG. 1B, the inlet valve 122 is coupled to the canister 302 and the inlet valve 124 is coupled to the canister 304. Outlet valve 132 is coupled to the canister 302 and outlet valve 134 is coupled to canister 304. The inlet valves 122 and 124 are used to control the passage of air from the compression system 200 to the respective canisters. The outlet valves 132 and 134 are used to release gas from the respective canisters 302 and 304 during a venting process. In some implementations, the inlet valves 122 and 124 and the outlet valves 132 and 134 may be silicon plunger solenoid valves. Other types of valves, however, may be used. Plunger valves offer advantages over other kinds of valves by being quiet and having low slippage.

In some implementations, a two-step valve actuation voltage may be generated to control the inlet valves 122 and 124 and the outlet valves 132 and 134. For example, a high voltage (e.g., 24 V) may be applied to an inlet valve to open the inlet valve. The voltage may then be reduced (e.g., to 7 V) to keep the inlet valve open. Using less voltage to keep a valve open may use less power. This reduction in voltage minimizes heat buildup and power consumption to extend run time from the power supply 180 (described below). When the power is cut off to the valve, it closes by spring action. In some implementations, the voltage may be applied as a function of time that is not necessarily a stepped response (e.g., a curved downward voltage between an initial 24 V and a final 7 V).

In an implementation, a controller 400 is electrically coupled to valves 122, 124, 132, and 134. The controller 400 includes one or more processors 410 operable to execute program instructions stored in a memory 420. The program instructions configure the controller 400 to perform various predefined methods that are used to operate the oxygen concentrator, such as the methods described in more detail herein. The program instructions may include program instructions for operating the inlet valves 122 and 124 out of phase with each other, i.e., when one of the inlet valves 122 or 124 is opened, the other valve is closed such as when electro-mechanical valve(s) are used. During pressurization of the canister 302, the outlet valve 132 is closed and the outlet valve 134 is opened. Similar to the inlet valves, the outlet valves 132 and 134 are operated out of phase with each other. In some implementations, the voltages and the durations of the voltages used to open the input and output valves may be controlled by controller 400.

Check valves 142 and 144 are coupled to canisters 302 and 304, respectively. Check valves 142 and 144 are one-way valves that are passively operated by the pressure differentials that occur as the canisters are pressurized and vented, or may be active valves. Check valves 142 and 144 are coupled to the canisters to allow oxygen enriched air produced during pressurization of each canister to flow out of the canister, and to inhibit back flow of oxygen enriched air or any other gases into the canister. In this manner, check valves 142 and 144 act as one-way valves allowing oxygen enriched air to exit the respective canisters during pressurization.

The term “check valve,” as used herein, refers to a valve that allows flow of a fluid (gas or liquid) in one direction and inhibits back flow of the fluid. Examples of check valves that are suitable for use include, but are not limited to: a ball check valve; a diaphragm check valve; a butterfly check valve; a swing check valve; a duckbill valve; an umbrella valve; and a lift check valve. Under pressure, nitrogen molecules in the pressurized ambient air are adsorbed by the gas separation adsorbent in the pressurized canister. As the pressure increases, more nitrogen is adsorbed until the gas in the canister is enriched in oxygen. The non-adsorbed gas molecules (mainly oxygen) flow out of the pressurized canister when the pressure reaches a point sufficient to overcome the resistance of the check valve coupled to the canister. In one implementation, the pressure drop of the check valve in the forward direction is less than 1 psi (6.9 kPa). The break pressure in the reverse direction is greater than 100 psi (689.5 kPa). It should be understood, however, that modification of one or more components would alter the operating parameters of these valves. If the forward flow pressure is increased, there is, generally, a reduction in oxygen enriched air production. If the break pressure for reverse flow is reduced or set too low, there is, generally, a reduction in oxygen enriched air pressure.

In an exemplary implementation, the canister 302 is pressurized by compressed air produced in compression system 200 and passed into the canister 302. During pressurization of the canister 302, the inlet valve 122 is open, the outlet valve 132 is closed, the inlet valve 124 is closed and outlet valve 134 is open. The outlet valve 134 is opened when outlet valve 132 is closed to allow substantially simultaneous venting of canister 304 to atmosphere while canister 302 is being pressurized.

After some time, the pressure in canister 302 is sufficient to open check valve 142. Oxygen enriched air produced in canister 302 passes through check valve 142 and, in one implementation, is collected in an accumulator 106.

After some further time, the gas separation adsorbent in canister 302 becomes saturated with nitrogen and is unable to separate significant amounts of nitrogen from incoming air. This point is usually reached after a predetermined time of oxygen enriched air production. In the implementation described above, when the gas separation adsorbent in the canister 302 reaches this saturation point, the inflow of compressed air is stopped and the canister 302 is vented to desorb nitrogen. During venting of canister 302, the inlet valve 122 is closed, and the outlet valve 132 is opened. While the canister 302 is being vented, the canister 304 is pressurized to produce oxygen enriched air in the same manner described above. Pressurization of the canister 304 is achieved by closing the outlet valve 134 and opening the inlet valve 124. After some time, the oxygen enriched air exits the canister 304 through the check valve 144.

During venting of the canister 302, the outlet valve 132 is opened allowing exhaust gas (mainly nitrogen) to exit the canister 302 to atmosphere through the concentrator outlet 130. In an implementation, the vented exhaust gas may be directed through the muffler 133 to reduce the noise produced by releasing the pressurized gas from the canister. As exhaust gas is vented from the canister 302, the pressure in the canister 302 drops, allowing the nitrogen to become desorbed from the gas separation adsorbent. The desorption of the nitrogen resets the canister 302 to a state that allows renewed separation of nitrogen from an air stream. The muffler 133 may include open cell foam (or another material) to muffle the sound of the gas leaving the oxygen concentrator 100. In some implementations, the combined muffling components/techniques for the input of air and the output of oxygen enriched air may provide for oxygen concentrator operation at a sound level below 50 decibels.

During venting of the canisters 302 and 304, it is advantageous that at least a majority of the nitrogen is removed. In an implementation, at least about 50%, at least about 60%, at least about 70%, at least about 80%, at least about 90%, at least about 95%, at least about 98%, or substantially all of the nitrogen in a canister is removed before the canister is re-used to separate nitrogen from air.

In some implementations, nitrogen removal may be assisted using an oxygen enriched air stream that is introduced into the canister from the other canister or stored oxygen enriched air. In an exemplary implementation, a portion of the oxygen enriched air may be transferred from the canister 302 to the canister 304 when the canister 304 is being vented of exhaust gas. Transfer of oxygen enriched air from canister 302 to canister 304 during venting of canister 304 helps to desorb nitrogen from the adsorbent by lowering the partial pressure of nitrogen adjacent the adsorbent. The flow of oxygen enriched air also helps to purge desorbed nitrogen (and other gases) from the canister. In an implementation, oxygen enriched air may travel through flow restrictors 151, 153, and 155 between the two canisters 302 and 304. The flow restrictor 151 may be a trickle flow restrictor. The flow restrictor 151, for example, may be a 0.009D flow restrictor (e.g., the flow restrictor has a radius 0.009″ (0.022 cm) which is less than the diameter of the tube it is inside). Flow restrictors 153 and 155 may be 0.013D flow restrictors. Other flow restrictor types and sizes are also contemplated and may be used depending on the specific configuration and tubing used to couple the canisters. In some implementations, the flow restrictors may be press fit flow restrictors that restrict air flow by introducing a narrower diameter in their respective conduits. In some implementations, the press fit flow restrictors may be made of sapphire, metal or plastic (other materials are also contemplated).

Flow of oxygen enriched air between the canisters may also be controlled by use of a valve 152 and a valve 154. The valves 152 and 154 may be opened for a short duration during the venting process (and may be closed otherwise) to prevent excessive oxygen loss out of the purging canister. Other durations are also contemplated. In an exemplary implementation, the canister 302 is vented and it is desirable to purge the canister 302 by passing a portion of the oxygen enriched air produced in the canister 304 into the canister 302. A portion of oxygen enriched air, upon pressurization of the canister 304, will pass through a flow restrictor 151 into the canister 302 during venting of the canister 302. Additional oxygen enriched air is passed into the canister 302, from the canister 304, through the valve 154 and the flow restrictor 155. The valve 152 may remain closed during the transfer process, or may be opened if additional oxygen enriched air is needed. The selection of appropriate flow restrictors 151 and 155, coupled with controlled opening of the valve 154 allows a controlled amount of oxygen enriched air to be sent from the canister 304 to the canister 302. In an implementation, the controlled amount of oxygen enriched air is an amount sufficient to purge the canister 302 and minimize the loss of oxygen enriched air through venting the valve 132 of the canister 302. While this implementation describes venting of the canister 302, it should be understood that the same process can be used to vent the canister 304 using the flow restrictor 151, the valve 152 and the flow restrictor 153.

The pair of equalization/vent valves 152 and 154 work with the flow restrictors 153 and 155 to optimize the gas flow balance between the two canisters 302 and 304. This may allow for better flow control for purging one of the canisters 302 and 304 with oxygen enriched air from the other of the canisters. It may also provide better flow direction between the two canisters 302 and 304. While the flow valves 152 and 154 may be operated as bi-directional valves, the flow rate through such valves varies depending on the direction of fluid flowing through the valve. For example, oxygen enriched air flowing from the canister 304 toward the canister 302 has a flow rate faster through the valve 152 than the flow rate of oxygen enriched air flowing from the canister 302 toward the canister 304 through the valve 152. If a single valve was to be used, eventually either too much or too little oxygen enriched air would be sent between the canisters and the canisters would, over time, begin to produce different amounts of oxygen enriched air. Use of opposing valves and flow restrictors on parallel air pathways may equalize the flow pattern of the oxygen enriched air between the two canisters. Equalizing the flow may allow for a steady amount of oxygen enriched air to be available to the user over multiple cycles and also may allow a predictable volume of oxygen enriched air to purge the other of the canisters. In some implementations, the air pathway may not have restrictors but may instead have a valve with a built-in resistance or the air pathway itself may have a narrow radius to provide resistance.

At times, the oxygen concentrator 100 may be shut down for a period of time. When an oxygen concentrator is shut down, the temperature inside the canisters may drop as a result of the loss of adiabatic heat from the compression system. As the temperature drops, the volume occupied by the gases inside the canisters will drop. Cooling of the canisters 302 and 304 may lead to a negative pressure in the canisters 302 and 304. Valves (e.g., valves 122, 124, 132, and 134) leading to and from the canisters 302 and 304 are dynamically sealed rather than hermetically sealed. Thus, outside air may enter the canisters 302 and 304 after shutdown to accommodate the pressure differential. When outside air enters the canisters 302 and 304, moisture from the outside air may be adsorbed by the gas separation adsorbent. Adsorption of water inside the canisters 302 and 304 may lead to gradual degradation of the gas separation adsorbents, steadily reducing ability of the gas separation adsorbents to produce oxygen enriched air.

In an implementation, outside air may be inhibited from entering the canisters 302 and 304 after the oxygen concentrator 100 is shut down by pressurizing both canisters 302 and 304 prior to shutdown. By storing the canisters 302 and 304 under a positive pressure, the valves may be forced into a hermetically closed position by the internal pressure of the air in the canisters 302 and 304. In an implementation, the pressure in the canisters, 302 and 304 at shutdown, should be at least greater than ambient pressure. As used herein the term “ambient pressure” refers to the pressure of the surroundings in which the oxygen concentrator 100 is located (e.g. the pressure inside a room, outside, in a plane, etc.). In an implementation, the pressure in the canisters 302 and 304, at shut down, is at least greater than standard atmospheric pressure (i.e., greater than 760 mmHg (Torr), 1 atm, 101,325 Pa). In an implementation, the pressure in the canisters 302 and 304, at shutdown, is at least about 1.1 times greater than ambient pressure; is at least about 1.5 times greater than ambient pressure; or is at least about 2 times greater than ambient pressure.

In an implementation, pressurization of the canisters 302 and 304 may be achieved by directing pressurized air into each canister 302 and 304 from the compression system and closing all valves to trap the pressurized air in the canisters. In an exemplary implementation, when a shutdown sequence is initiated, inlet valves 122 and 124 are opened and outlet valves 132 and 134 are closed. Because the inlet valves 122 and 124 are joined together by a common conduit, both canisters 302 and 304 may become pressurized as air and/or oxygen enriched air from one canister may be transferred to the other canister. This situation may occur when the pathway between the compression system and the two inlet valves allows such transfer. Because the oxygen concentrator 100 operates in an alternating pressurize/venting mode, at least one of the canisters 302 and 304 should be in a pressurized state at any given time. In an alternate implementation, the pressure may be increased in each canister 302 and 304 by operation of compression system 200. When the inlet valves 122 and 124 are opened, pressure between the canisters 302 and 304 will equalize, however, the equalized pressure in either canister may not be sufficient to inhibit air from entering the canisters during shutdown. In order to ensure that air is inhibited from entering the canisters, compression system 200 may be operated for a time sufficient to increase the pressure inside both canisters to a level at least greater than ambient pressure. Regardless of the method of pressurization of the canisters, once the canisters are pressurized, inlet valves 122 and 124 are closed, trapping the pressurized air inside the canisters, which inhibits air from entering the canisters during the shutdown period.

Referring to FIG. 1C, an implementation of an oxygen concentrator 100 is depicted. In this example, the oxygen concentrator 100 includes a compression system 200, a canister system 300, and a power supply 180 disposed within an outer housing 170. Inlets 101 are located in outer housing 170 to allow air from the environment to enter oxygen concentrator 100. The inlets 101 may allow air to flow into the compartment to assist with cooling of the components in the compartment. A power supply 180 provides a source of power for the oxygen concentrator 100. The compression system 200 draws air in through the inlet 105 and a muffler 108. The muffler 108 may reduce noise of air being drawn in by the compression system and also may include a desiccant material to remove moisture, i.e. water, from the incoming air. The oxygen concentrator 100 may further include a fan 172 used to vent air and other gases from the oxygen concentrator via the outlet 173.

In some implementations, the compression system 200 includes one or more compressors. In another implementation, the compression system 200 includes a single compressor, coupled to all of the canisters of the canister system 300. Turning to FIGS. 1D and 1E, a compression system 200 is depicted that includes a compressor 210 and a motor 220. The motor 220 is coupled to the compressor 210 and provides an operating force to the compressor 210 to operate the compression mechanism. For example, the motor 220 may be a motor providing a rotating component that causes cyclical motion of a component of the compressor 210 that compresses air. When the compressor 210 is a piston type compressor, the motor 220 provides an operating force which causes the piston of the compressor 210 to be reciprocated. Reciprocation of the piston causes compressed air to be produced by the compressor 210. The pressure of the compressed air is, in part, estimated by the speed that the compressor is operated at, (e.g., how fast the piston is reciprocated). The motor 220, therefore, may be a variable speed motor that is operable at various speeds to dynamically control the pressure of air produced by the compressor 210.

In one implementation, the compressor 210 includes a single head wobble type compressor having a piston. Other types of compressors may be used such as diaphragm compressors and other types of piston compressors. The motor 220 may be a DC or AC motor and provides the operating power to the compressing component of the compressor 210. The motor 220, in an implementation, may be a brushless DC motor. The motor 220 may be a variable speed motor configured to operate the compressing component of the compressor 210 at variable speeds. The motor 220 may be coupled to a controller 400, as depicted in FIG. 1B, which sends operating signals to the motor to control the operation of the motor. For example, the controller 400 may send signals to the motor 220 to: turn the motor on, turn motor the off, and set the operating speed of the motor. Thus, as illustrated in FIG. 1B, the compression system 200 may include a speed sensor 201. The speed sensor 201 may be a motor speed transducer used to determine a rotational speed of the motor 220 and/or a frequency of another reciprocating operation of the compression system 200. For example, a motor speed signal from the motor speed transducer may be provided to the controller 400. The speed sensor or motor speed transducer may, for example, be a Hall effect sensor. The controller 400 may operate the compression system via the motor 220 based on the speed signal and/or any other sensor signal of the oxygen concentrator, such as a pressure sensor (e.g., accumulator pressure sensor 107). As illustrated in FIG. 1B, the controller 400 receives sensor signals, such as a speed signal from the speed sensor 201 and an accumulator pressure signal from the accumulator pressure sensor 107. With such signal(s), the controller may implement one or more control loops (e.g., feedback control) for operation of the compression system based on sensor signals such as accumulator pressure and/or motor speed as described in more detail herein.

The compression system 200 inherently creates substantial heat. Heat is caused by the consumption of power by the motor 220 and the conversion of power into mechanical motion. The compressor 210 generates heat due to the increased resistance to movement of the compressor components by the air being compressed. Heat is also inherently generated due to adiabatic compression of the air by the compressor 210. Thus, the continual pressurization of air produces heat in the enclosure. Additionally, the power supply 180 may produce heat as power is supplied to the compression system 200. Furthermore, users of the oxygen concentrator may operate the device in unconditioned environments (e.g., outdoors) at potentially higher ambient temperatures than indoors, thus the incoming air will already be in a heated state.

Heat produced inside the oxygen concentrator 100 can be problematic. Lithium ion batteries are generally employed as power supplies for oxygen concentrators due to their long life and light weight. Lithium ion battery packs, however, are dangerous at elevated temperatures and safety controls are employed in the oxygen concentrator 100 to shut down the system if dangerously high power supply temperatures are detected. Additionally, as the internal temperature of the oxygen concentrator 100 increases, the amount of oxygen generated by the concentrator may decrease. This is due, in part, to the decreasing amount of oxygen in a given volume of air at higher temperatures. If the amount of produced oxygen drops below a predetermined amount, the oxygen concentrator 100 may automatically shut down.

Because of the compact nature of oxygen concentrators, dissipation of heat can be difficult. Solutions typically involve the use of one or more fans to create a flow of cooling air through the enclosure. Such solutions, however, require additional power from the power supply 180 and thus shorten the portable usage time of the oxygen concentrator 100. In an implementation, a passive cooling system may be used that takes advantage of the mechanical power produced by the motor 220. Referring to FIGS. 1D and 1E, the motor 220 of the compression system 200 has an external rotatable armature 230. Specifically, the armature 230 of the motor 220 (e.g. a DC motor) is wrapped around the stationary field that is driving the armature 230. Since the motor 220 is a large contributor of heat to the overall system it is helpful to transfer heat off the motor and sweep it out of the enclosure. With the external high speed rotation, the relative velocity of the major component of the motor 220 and the air in which it exists is very high. The surface area of the armature is larger if externally mounted than if it is internally mounted. Since the rate of heat exchange is proportional to the surface area and the square of the velocity, using a larger surface area armature mounted externally increases the ability of heat to be dissipated from the motor 220. The gain in cooling efficiency by mounting the armature 230 externally, allows the elimination of one or more cooling fans, thus reducing the weight and power consumption while maintaining the interior of the oxygen concentrator within the appropriate temperature range. Additionally, the rotation of the externally mounted armature 230 creates movement of air proximate to the motor to create additional cooling.

Moreover, an external rotating armature may help the efficiency of the motor, allowing less heat to be generated. A motor having an external armature operates similar to the way a flywheel works in an internal combustion engine. When the motor is driving the compressor, the resistance to rotation is low at low pressures. When the pressure of the compressed air is higher, the resistance to rotation of the motor is higher. As a result, the motor does not maintain consistent ideal rotational stability, but instead surges and slows down depending on the pressure demands of the compressor. This tendency of the motor to surge and then slow down is inefficient and therefore generates heat. Use of an external armature adds greater angular momentum to the motor which helps to compensate for the variable resistance experienced by the motor. Since the motor does not have to work as hard, the heat produced by the motor may be reduced.

In an implementation, cooling efficiency may be further increased by coupling an air transfer device 240 to the external rotating armature 230. In an implementation, the air transfer device 240 is coupled to the external armature 230 such that rotation of the external armature 230 causes the air transfer device 240 to create an air flow that passes over at least a portion of the motor. In an implementation, the air transfer device 240 includes one or more fan blades coupled to the external armature 230. In an implementation, a plurality of fan blades may be arranged in an annular ring such that the air transfer device 240 acts as an impeller that is rotated by movement of the external rotating armature 230. As depicted in FIGS. 1D and 1E, the air transfer device 240 may be mounted to an outer surface of the external armature 230, in alignment with the motor 220. The mounting of the air transfer device 240 to the armature 230 allows air flow to be directed toward the main portion of the external rotating armature 230, providing a cooling effect during use. In an implementation, the air transfer device 240 directs air flow such that a majority of the external rotating armature 230 is in the air flow path.

Further, referring to FIGS. 1D and 1E, air pressurized by the compressor 210 exits the compressor 210 at the compressor outlet 212. A compressor outlet conduit 250 is coupled to the compressor outlet 212 to transfer the compressed air to the canister system 300. As noted previously, compression of air causes an increase in the temperature of the air. This increase in temperature can be detrimental to the efficiency of the oxygen concentrator. In order to reduce the temperature of the pressurized air, the compressor outlet conduit 250 is placed in the air flow path produced by the air transfer device 240. At least a portion of the compressor outlet conduit 250 may be positioned proximate to the motor 220. Thus, air flow, created by air transfer device, may contact both the motor 220 and the compressor outlet conduit 250. In one implementation, a majority of the compressor outlet conduit 250 is positioned proximate to the motor 220. In an implementation, the compressor outlet conduit 250 is coiled around the motor 220, as depicted in FIG. 1E.

In an implementation, the compressor outlet conduit 250 is composed of a heat exchange metal. Heat exchange metals include, but are not limited to, aluminum, carbon steel, stainless steel, titanium, copper, copper-nickel alloys or other alloys formed from combinations of these metals. Thus, the compressor outlet conduit 250 can act as a heat exchanger to remove heat that is inherently caused by compression of the air. By removing heat from the compressed air, the number of molecules in a given volume at a given pressure is increased. As a result, the amount of oxygen enriched air that can be generated by each canister during each PSA cycle may be increased.

The heat dissipation mechanisms described herein are either passive or make use of elements required for the oxygen concentrator 100. Thus, for example, dissipation of heat may be increased without using systems that require additional power. By not requiring additional power, the run-time of the battery packs may be increased and the size and weight of the oxygen concentrator may be minimized. Likewise, use of an additional box fan or cooling unit may be eliminated. Eliminating such additional features reduces the weight and power consumption of the oxygen concentrator.

As discussed above, adiabatic compression of air causes the air temperature to increase. During venting of a canister in the canister system 300, the pressure of the exhaust gas being vented from the canisters decreases. The adiabatic decompression of the gas in the canister causes the temperature of the gas to drop as it is vented. In an implementation, the cooled exhaust gas 327 vented from the canister system 300 is directed toward the power supply 180 and toward the compression system 200. In an implementation, a base 315 of canister system 300 receives the exhaust gas 327 from the canisters. The exhaust gas 327 is directed through the base 315 toward the outlet 325 of the base and toward the power supply 180. The exhaust gas, as noted, is cooled due to decompression of the gases and therefore passively provide cooling to the power supply. When the compression system is operated, the air transfer device will gather the cooled exhaust gas and direct the exhaust gas 327 toward the motor 220 of the compression system 200. The fan 172 may also assist in directing the exhaust gas 327 across the compression system 200 and out of the housing 170. In this manner, additional cooling may be obtained without requiring any further power requirements from the battery.

The oxygen concentrator 100 may include at least two canisters, each canister including a gas separation adsorbent. The canisters of the oxygen concentrator 100 may be formed from a molded housing. In an implementation, the prior art canister system 300 (aka sieve bed) includes two housing components 310 and 510, as depicted in FIG. 1I. In various implementations, the housing components 310 and 510 of the oxygen concentrator 100 may form a two-part molded plastic frame that defines the two canisters 302 and 304 and the accumulator 106.

The housing components 310 and 510 may be formed separately and then coupled together. In some implementations, the housing components 310 and 510 may be injection molded or compression molded. The housing components 310 and 510 may be made from a thermoplastic polymer such as polycarbonate, methylene carbide, polystyrene, acrylonitrile butadiene styrene (ABS), polypropylene, polyethylene, or polyvinyl chloride. In another implementation, the housing components 310 and 510 may be made of a thermoset plastic or metal (such as stainless steel or a lightweight aluminum alloy). Lightweight materials may be used to reduce the weight of the oxygen concentrator 100. In some implementations, the two housing components 310 and 510 may be fastened together using screws or bolts. Alternatively, the housing components 310 and 510 may be solvent welded together.

As shown, the valve seats 322, 324, 332, and 334 and conduits 330 and 346 may be integrated into the housing component 310 to reduce the number of sealed connections needed throughout the air flow of the oxygen concentrator 100.

Air pathways/tubing between different sections in the housing components 310 and 510 may take the form of molded conduits. Conduits in the form of molded channels for air pathways may occupy multiple planes in the housing components 310 and 510. For example, the molded air conduits may be formed at different depths and at different positions in the housing components 310 and 510. In some implementations, a majority or substantially all of the conduits may be integrated into the housing components 310 and 510 to reduce potential leak points.

In some implementations, prior to coupling the housing components 310 and 510 together, O-rings may be placed between various points of the housing components 310 and 510 to ensure that the housing components are properly sealed. In some implementations, components may be integrated and/or coupled separately to the housing components 310 and 510. For example, tubing, flow restrictors (e.g., press fit flow restrictors), oxygen sensors, gas separation adsorbents, check valves, plugs, processors, power supplies, etc. may be coupled to the housing components 310 and 510 before and/or after the housing components are coupled together.

In some implementations, apertures 337 leading to the exterior of the housing components 310 and 510 may be used to insert devices such as flow restrictors. Apertures may also be used for increased moldability. One or more of the apertures may be plugged after molding (e.g., with a plastic plug). In some implementations, flow restrictors may be inserted into passages prior to inserting plugs to seal the passages. Press fit flow restrictors may have diameters that may allow a friction fit between the press fit flow restrictors and their respective apertures. In some implementations, an adhesive may be added to the exterior of the press fit flow restrictors to hold the press fit flow restrictors in place once inserted. In some implementations, the plugs may have a friction fit with their respective tubes (or may have an adhesive applied to their outer surface). The press fit flow restrictors and/or other components may be inserted and pressed into their respective apertures using a narrow tip tool or rod (e.g., with a diameter less than the diameter of the respective aperture). In some implementations, the press fit flow restrictors may be inserted into their respective tubes until they abut a feature in the tube to halt their insertion. For example, the feature may include a reduction in radius. Other features are also contemplated (e.g., a bump in the side of the tubing, threads, etc.). In some implementations, press fit flow restrictors may be molded into the housing components (e.g., as narrow tube segments).

In some implementations, a spring baffle 139 may be placed into respective canister receiving portions of the housing components 310 and 510 with the spring side of the baffle 139 facing the exit of the canister. The spring baffle 139 may apply force to gas separation adsorbent in the canister while also assisting in preventing gas separation adsorbent from entering the exit apertures. Use of the spring baffle 139 may keep the gas separation adsorbent compact while also allowing for expansion (e.g., thermal expansion). Keeping the gas separation adsorbent compact may prevent the gas separation adsorbent from breaking during movement of the oxygen concentrator 100.

In some implementations, a filter 129 may be placed into respective canister receiving portions of the housing components 310 and 510 facing the inlet of the respective canisters. The filter 129 removes particles from the feed gas stream entering the canisters.

In some implementations, pressurized air from the compression system 200 may enter an air inlet 306. The air inlet 306 is coupled to an inlet conduit 330. Air enters the housing component 310 through the inlet 306, travels through the inlet conduit 330, and then to the valve seats 322 and 324. FIG. 1J and FIG. 1K depict an end view of the housing 310. FIG. 1J depicts an end view of the housing 310 prior to fitting valves to the housing component 310. FIG. 1K depicts an end view of the housing component 310 with the valves fitted to the housing component 310. The valve seats 322 and 324 are configured to receive the inlet valves 122 and 124 respectively. The inlet valve 122 is coupled to the canister 302 and the inlet valve 124 is coupled to the canister 304. The housing component 310 also includes the valve seats 332 and 334 configured to receive the outlet valves 132 and 134 respectively. The outlet valve 132 is coupled to the canister 302 and the outlet valve 134 is coupled to the canister 304. The inlet valves 122 and 124 are used to control the passage of air from the inlet conduit 330 to the respective canisters.

In an implementation, pressurized air is sent into one of canisters 302 or 304 while the other canister is being vented. Valve seat 322 includes an opening 323 that passes through the housing component 310 into the canister 302. Similarly, the valve seat 324 includes an opening 375 that passes through the housing component 310 into the canister 304. Air from the inlet conduit 330 passes through the openings 323 or 375 if the respective valves 122 and 124 are open, and enters the respective canisters 302 and 304.

Check valves 142 and 144 (See FIG. 1I) are coupled to the canisters 302 and 304, respectively. The check valves 142 and 144 are one way valves that are passively operated by the pressure differentials that occur as the canisters are pressurized and vented. Oxygen enriched air produced in the canisters 302 and 304 passes from the canisters into the openings 542 and 544 of the housing component 510. A passage (not shown) links the openings 542 and 544 to the conduits 342 and 344, respectively. Oxygen enriched air produced in the canister 302 passes from the canister 302 though the opening 542 and into the conduit 342 when the pressure in the canister 302 is sufficient to open the check valve 142. When the check valve 142 is open, oxygen enriched air flows through the conduit 342 toward the end of the housing component 310. Similarly, oxygen enriched air produced in the canister 304 passes from the canister 304 through the opening 544 and into the conduit 344 when the pressure in the canister 304 is sufficient to open the check valve 144. When the check valve 144 is open, oxygen enriched air flows through the conduit 344 toward the end of the housing component 310.

Oxygen enriched air from either canister 302 or 304 travels through the conduit 342 or 344 and enters the conduit 346 formed in the housing component 310. The conduit 346 includes openings that couple the conduit to the conduit 342, the conduit 344 and the accumulator 106. Thus, oxygen enriched air, produced in the canister 302 or 304, travels to conduit 346 and passes into the accumulator 106. As illustrated in FIG. 1B, gas pressure within the accumulator 106 may be measured by a sensor, such as with an accumulator pressure sensor 107. (See also FIG. 1F.) Thus, the accumulator pressure sensor 107 generates a signal representing the pressure of the accumulated oxygen enriched air. An example of a suitable pressure transducer is a sensor from the HONEYWELL ASDX series. An alternative suitable pressure transducer is a sensor from the NPA Series from GENERAL ELECTRIC. In some versions, the pressure sensor 107 may alternatively measure pressure of the gas outside of the accumulator 106, such as in an output path between the accumulator 106 and a valve (e.g., supply valve 160) that controls the release of the oxygen enriched air for delivery to a user in a bolus.

The canister 302 is vented by closing the inlet valve 122 and opening the outlet valve 132. The outlet valve 132 releases the exhaust gas from the canister 302 into the volume defined by the end of the housing component 310. Foam material may cover the end of the housing component 310 to reduce the sound made by release of gases from the canisters. Similarly, the canister 304 is vented by closing the inlet valve 124 and opening outlet the valve 134. The outlet valve 134 releases the exhaust gas from the canister 304 into the volume defined by the end of the housing component 310.

Three conduits are formed in the housing component 510 for use in transferring oxygen enriched air between the canisters 302 and 304. As shown in FIG. 1L, the conduit 530 couples the canister 302 to the canister 304. The flow restrictor 151 (not shown) is disposed in the conduit 530, between the canister 302 and the canister 304 to restrict flow of oxygen enriched air during use. The conduit 532 also couples the canister 302 to the canister 304. The conduit 532 is coupled to the valve seat 552 which receives the valve 152, as shown in FIG. 1M. The flow restrictor 153 (not shown) is disposed in the conduit 532, between the canister 302 and the canister 304. The conduit 534 also couples the canister 302 to the canister 304. The conduit 534 is coupled to the valve seat 554 which receives the valve 154, as shown in FIG. 1M. The flow restrictor 155 (not shown) is disposed in the conduit 534, between the canister 302 and the canister 304. The pair of equalization/vent valves 152/154 work with the flow restrictors 153 and 155 to optimize the air flow balance between the two canisters 302 and 304.

Oxygen enriched air in the accumulator 106 passes through the supply valve 160 into the expansion chamber 162 which is formed in the housing component 510. An opening (not shown) in the housing component 510 couples accumulator 106 to the supply valve 160. In an implementation, the expansion chamber 162 may include one or more devices configured to estimate an oxygen concentration of gas passing through the chamber.

An outlet system, coupled to one or more of the canisters, includes one or more conduits for providing oxygen enriched air to a user. In an implementation, oxygen enriched air produced in either of the canisters 302 and 304 is collected in the accumulator 106 through the check valves 142 and 144, respectively, as depicted schematically in FIG. 1B. The oxygen enriched air leaving the canisters 302 and 304 may be collected in the oxygen accumulator 106 prior to being provided to a user. In some implementations, a conduit may be coupled to the accumulator 106 to provide the oxygen enriched air to the user. Oxygen enriched air may be provided to the user through an airway delivery device (e.g., a patient interface) that transfers the oxygen enriched air to the user's mouth and/or nose. In an implementation, a delivery device may include a tube that directs the oxygen toward a user's nose and/or mouth that may not be directly coupled to the user's nose.

Turning to FIG. 1F, a schematic diagram of an implementation of an outlet system for an oxygen concentrator is shown. A supply valve 160 may be coupled to a conduit to control the release of the oxygen enriched air from the accumulator 106 to the user. In an implementation, supply valve 160 is an electromagnetically actuated plunger valve. The supply valve 160 is actuated by the controller 400 to control the delivery of oxygen enriched air to a user. Actuation of supply valve 160 is not timed or synchronized to the pressure swing adsorption process. Instead, actuation is synchronized to the user's breathing as described below. In some implementations, the supply valve 160 may have continuously-valued actuation to establish a clinically effective amplitude profile for providing oxygen enriched air.

Oxygen enriched air in the accumulator 106 passes through the supply valve 160 into the expansion chamber 162 as depicted in FIG. 1F. In an implementation, the expansion chamber 162 may include one or more devices configured to estimate an oxygen concentration of gas passing through the expansion chamber 162. Oxygen enriched air in the expansion chamber 162 builds briefly, through release of gas from the accumulator 106 by the supply valve 160, and then is bled through a small orifice flow restrictor 175 to a flow rate sensor 185 and then to a particulate filter 187. The flow restrictor 175 may be a 0.025 D flow restrictor. Other flow restrictor types and sizes may be used. In some implementations, the diameter of the air pathway in the housing may be restricted to create restricted gas flow. The flow rate sensor 185 may be any sensor configured to generate a signal representing the rate of gas flowing through the conduit. A particulate filter 187 may be used to filter bacteria, dust, granule particles, etc., prior to delivery of the oxygen enriched air to the user. The oxygen enriched air passes through the filter 187 to the connector 190 which sends the oxygen enriched air to the user via the delivery conduit 192 and to the pressure sensor 194.

The fluid dynamics of the outlet pathway, coupled with the programmed actuations of the supply valve 160, may result in a bolus of oxygen being provided at the correct time and with an amplitude profile that assures rapid delivery into the user's lungs without excessive waste.

The expansion chamber 162 may include one or more oxygen sensors adapted to determine an oxygen concentration of gas passing through the chamber. In an implementation, the oxygen concentration of gas passing through the expansion chamber 162 is estimated using an oxygen sensor 165. An oxygen sensor is a device configured to measure oxygen concentration in a gas. Examples of oxygen sensors include, but are not limited to, ultrasonic oxygen sensors, electrical oxygen sensors, chemical oxygen sensors, and optical oxygen sensors. In one implementation, the oxygen sensor 165 is an ultrasonic oxygen sensor that includes an ultrasonic emitter 166 and an ultrasonic receiver 168. In some implementations, the ultrasonic emitter 166 may include multiple ultrasonic emitters and the ultrasonic receiver 168 may include multiple ultrasonic receivers. In implementations having multiple emitters/receivers, the multiple ultrasonic emitters and multiple ultrasonic receivers may be axially aligned (e.g., across the gas flow path which may be perpendicular to the axial alignment).

In use, the ultrasonic sound wave from emitter 166 may be directed through oxygen enriched air disposed in the chamber 162 to the receiver 168. The ultrasonic oxygen sensor 165 may be configured to detect the speed of sound through the oxygen enriched air to determine the composition of the oxygen enriched air. The speed of sound is different in nitrogen and oxygen, and in a mixture of the two gases, the speed of sound through the mixture may be an intermediate value proportional to the relative amounts of each gas in the mixture. In use, the sound at the receiver 168 is slightly out of phase with the sound sent from the emitter 166. This phase shift is due to the relatively slow velocity of sound through a gas medium as compared with the relatively fast speed of the electronic pulse through wire. The phase shift, then, is proportional to the distance between the emitter 166 and the receiver 168 and inversely proportional to the speed of sound through the expansion chamber 162. The density of the gas in the chamber 162 affects the speed of sound through the expansion chamber 162 and the density is proportional to the ratio of oxygen to nitrogen in the expansion chamber 162. Therefore, the phase shift can be used to measure the concentration of oxygen in the expansion chamber 162. In this manner the relative concentration of oxygen in the accumulator 106 may be estimated as a function of one or more properties of a detected sound wave traveling through the accumulator 106.

In some implementations, multiple emitters 166 and receivers 168 may be used. The readings from the emitters 166 and receivers 168 may be averaged to reduce errors that may be inherent in turbulent flow systems. In some implementations, the presence of other gases may also be detected by measuring the transit time and comparing the measured transit time to predetermined transit times for other gases and/or mixtures of gases.

The sensitivity of the ultrasonic oxygen sensor system may be increased by increasing the distance between the emitter 166 and the receiver 168, for example to allow several sound wave cycles to occur between the emitter 166 and the receiver 168. In some implementations, if at least two sound cycles are present, the influence of structural changes of the transducer may be reduced by measuring the phase shift relative to a fixed reference at two points in time. If the earlier phase shift is subtracted from the later phase shift, the shift caused by thermal expansion of the expansion chamber 162 may be reduced or cancelled. The shift caused by a change of the distance between the emitter 166 and the receiver 168 may be approximately the same at the measuring intervals, whereas a change owing to a change in oxygen concentration may be cumulative. In some implementations, the shift measured at a later time may be multiplied by the number of intervening cycles and compared to the shift between two adjacent cycles. Further details regarding sensing of oxygen in the expansion chamber may be found, for example, in U.S. patent application Ser. No. 12/163,549, entitled “Oxygen Concentrator Apparatus and Method”, which published as U.S. Publication No. 2009/0065007 A1 on Mar. 12, 2009 and is incorporated herein by reference.

The flow rate sensor 185 may be used to determine the flow rate of gas flowing through the outlet system. Flow rate sensors that may be used include, but are not limited to: diaphragm/bellows flow meters; rotary flow meters (e.g. Hall effect flow meters); turbine flow meters; orifice flow meters; and ultrasonic flow meters. The flow rate sensor 185 may be coupled to the controller 400. The rate of gas flowing through the outlet system may be an indication of the breathing volume of the user. Changes in the flow rate of gas flowing through the outlet system may also be used to determine a breathing rate of the user. The controller 400 may generate a control signal or trigger signal to control actuation of the supply valve 160. Such control of actuation of the supply valve may be based on the breathing rate and/or breathing volume of the user, as estimated by the flow rate sensor 185.

In some implementations, the ultrasonic sensor 165 and, for example, the flow rate sensor 185 may provide a measurement of an actual amount of oxygen being provided. For example, the flow rate sensor 185 may measure a volume of gas (based on flow rate) provided and the ultrasonic sensor 165 may provide the concentration of oxygen of the gas provided. These two measurements together may be used by the controller 400 to determine an approximation of the actual amount of oxygen provided to the user.

Oxygen enriched air passes through the flow rate sensor 185 to the filter 187. The filter 187 removes bacteria, dust, granule particles, etc. prior to providing the oxygen enriched air to the user. The filtered oxygen enriched air passes through the filter 187 to the connector 190. The connector 190 may be a “Y” connector coupling the outlet of the filter 187 to the pressure sensor 194 and the delivery conduit 192. The pressure sensor 194 may be used to monitor the pressure of the gas passing through the conduit 192 to the user. In some implementations, the pressure sensor 194 is configured to generate a signal that is proportional to the amount of positive or negative pressure applied to a sensing surface. Changes in pressure, sensed by the pressure sensor 194, may be used to determine a breathing rate of a user, as well as the onset of inhalation (also referred to as the trigger instant) as described below. The controller 400 may control actuation of the supply valve 160 based on the breathing rate and/or onset of inhalation of the user. In an implementation, the controller 400 may control actuation of the supply valve 160 based on information provided by either or both of the flow rate sensor 185 and the pressure sensor 194. The controller 400 may adjust the volume of each bolus by controlling the time the supply valve 160 is actuated. The controller 400 may calibrate the time of actuation and bolus volume by reading data from the ultrasonic sensor 165 and the flow rate sensor 185 so as to provide a measurement of the actual volume (dosage) of oxygen being provided.

Oxygen enriched air may be provided to a user through the delivery conduit 192. In an implementation, the delivery conduit 192 may be a silicone tube. The delivery conduit 192 may be coupled to a user using an airway delivery device 196, as depicted in FIGS. 1G and 1H. The airway delivery device 196 may be any device capable of providing the oxygen enriched air to nasal cavities or oral cavities. Examples of airway delivery devices include, but are not limited to: nasal masks, nasal pillows, nasal prongs, nasal cannulas, and mouthpieces. A nasal cannula airway delivery device 196 is depicted in FIG. 1G. The nasal cannula airway delivery device 196 is positioned proximate to a user's airway (e.g., proximate to the user's mouth and or nose) to allow delivery of the oxygen enriched air to the user while allowing the user to breathe air from the surroundings.

In an alternate implementation, a mouthpiece may be used to provide oxygen enriched air to the user. As shown in FIG. 1H, a mouthpiece 198 may be coupled to the oxygen concentrator 100. The mouthpiece 198 may be the only device used to provide oxygen enriched air to the user, or a mouthpiece may be used in combination with a nasal delivery device 196 (e.g., a nasal cannula). As depicted in FIG. 1H, oxygen enriched air may be provided to a user through both a nasal airway delivery device 196 and a mouthpiece 198.

The mouthpiece 198 is removably positionable in a user's mouth. In one implementation, the mouthpiece 198 is removably couplable to one or more teeth in a user's mouth. During use, oxygen enriched air is directed into the user's mouth via the mouthpiece. The mouthpiece 198 may be a night guard mouthpiece which is molded to conform to the user's teeth. Alternatively, mouthpiece may be a mandibular repositioning device. In an implementation, at least a majority of the mouthpiece is positioned in a user's mouth during use.

During use, oxygen enriched air may be directed to the mouthpiece 198 when a change in pressure is detected proximate to the mouthpiece. In one implementation, the mouthpiece 198 may be coupled to a pressure sensor 194. When a user inhales air through the user's mouth, the pressure sensor 194 may detect a drop in pressure proximate to the mouthpiece. The controller 400 of the oxygen concentrator 100 may control release of a bolus of oxygen enriched air to the user at the onset of inhalation.

During typical breathing of an individual, inhalation may occur through the nose, through the mouth or through both the nose and the mouth. Furthermore, breathing may change from one passageway to another depending on a variety of factors. For example, during more active activities, a user may switch from breathing through their nose to breathing through their mouth, or breathing through their mouth and nose. A system that relies on a single mode of delivery (either nasal or oral), may not function properly if breathing through the monitored pathway is stopped. For example, if a nasal cannula is used to provide oxygen enriched air to the user, an inhalation sensor (e.g., a pressure sensor or flow rate sensor) is coupled to the nasal cannula to determine the onset of inhalation. If the user stops breathing through their nose, and switches to breathing through their mouth, the oxygen concentrator 100 may not know when to provide the oxygen enriched air since there is no feedback from the nasal cannula. Under such circumstances, the oxygen concentrator 100 may increase the flow rate and/or increase the frequency of providing oxygen enriched air until the inhalation sensor detects an inhalation by the user. If the user switches between breathing modes often, the default mode of providing oxygen enriched air may cause the oxygen concentrator 100 to work harder, limiting the portable usage time of the system.

In an implementation, the mouthpiece 198 is used in combination with the nasal cannula airway delivery device 196 to provide oxygen enriched air to a user, as depicted in FIG. 1H. Both the mouthpiece 198 and the nasal airway delivery device 196 are coupled to an inhalation sensor. In one implementation, the mouthpiece 198 and the nasal airway delivery device 196 are coupled to the same inhalation sensor. In an alternate implementation, the mouthpiece 198 and the nasal cannula airway delivery device 196 are coupled to different inhalation sensors. In either implementation, the inhalation sensor(s) may detect the onset of inhalation from either the mouth or the nose. The oxygen concentrator 100 may be configured to provide oxygen enriched air to the delivery device (i.e., mouthpiece 198 or nasal cannula airway delivery device 196) proximate to which the onset of inhalation was detected. Alternatively, oxygen enriched air may be provided to both the mouthpiece 198 and the nasal airway delivery device 196 if onset of inhalation is detected proximate either delivery device. The use of a dual delivery system, such as depicted in FIG. 1H may be particularly useful for users when they are sleeping and may switch between nose breathing and mouth breathing without conscious effort.

Operation of oxygen concentrator 100 may be performed automatically using the internal controller 400 coupled to various components of the oxygen concentrator 100, as described herein. The controller 400 may include one or more processors 410, an internal memory 420, a cellular wireless module (CWM) module 430, and a GPS receiver 434 as depicted in FIG. 1B. Methods used to operate and monitor oxygen concentrator 100 may be implemented by program instructions stored in the internal memory 420 or an external memory medium coupled to the controller 400, and executed by one or more processors 410. A memory medium may include any of various types of memory devices or storage devices. The term “memory medium” is intended to include an installation medium, e.g., a Compact Disc Read Only Memory (CD-ROM), floppy disks, or tape device; a computer system memory or random access memory such as Dynamic Random Access Memory (DRAM), Double Data Rate Random Access Memory (DDR RAM), Static Random Access Memory (SRAM), Extended Data Out Random Access Memory (EDO RAM), Random Access Memory (RAM), etc.; or a non-volatile memory such as a magnetic medium, e.g., a hard drive, or optical storage. The memory medium may comprise other types of memory as well, or combinations thereof. In addition, the memory medium may be located proximate to the controller 400 by which the programs are executed, or may be located in an external computing device that connects to the controller 400 over a network, as described below. In the latter instance, the external computing device may provide program instructions to the controller 400 for execution. The term “memory medium” may include two or more memory media that may reside in different locations, e.g., in different computing devices that are connected over a network.

In some implementations, the controller 400 includes the processor 410 that includes, for example, one or more field programmable gate arrays (FPGAs), microcontrollers, etc. included on a circuit board disposed in the oxygen concentrator 100. The processor 410 is configured to execute programming instructions stored in the memory 420. In some implementations, programming instructions may be built into the processor 410 such that a memory external to the processor 410 may not be separately accessed (i.e., the memory 420 may be internal to the processor 410).

The processor 410 may be coupled to various components of oxygen concentrator 100, including, but not limited to the compression system 200, one or more of the valves used to control fluid flow through the system (e.g., valves 122, 124, 132, 134, 152, 154, 160), the oxygen sensor 165, the pressure sensor 194, the flow rate sensor 185, temperature sensors (not shown), the fan 172, the motor speed sensor 201, and any other component that may be electrically controlled. In some implementations, a separate processor (and/or memory) may be coupled to one or more of the components.

The controller 400 is configured (e.g., programmed by program instructions) to operate oxygen concentrator 100 and is further configured to monitor the oxygen concentrator 100 for malfunction states. For example, in one implementation, the controller 400 is programmed to trigger an alarm if the system is operating and no breathing is detected by the user for a predetermined amount of time. For example, if the controller 400 does not detect a breath for a period of 75 seconds, an alarm LED may be lit and/or an audible alarm may be sounded. If the user has truly stopped breathing, for example, during a sleep apnea episode, the alarm may be sufficient to awaken the user, causing the user to resume breathing. The action of breathing may be sufficient for the controller 400 to reset this alarm function. Alternatively, if the system is accidentally left on when the delivery conduit 192 is removed from the user, the alarm may serve as a reminder for the user to turn the oxygen concentrator 100 off.

The controller 400 is further coupled to the oxygen sensor 165, and may be programmed for continuous or periodic monitoring of the oxygen concentration of the oxygen enriched air passing through the expansion chamber 162. A minimum oxygen concentration threshold may be programmed into the controller 400, such that the controller 400 lights an LED visual alarm and/or an audible alarm to warn the user of the low concentration of oxygen.

The controller 400 is also coupled to the internal power supply 180 and may be configured to monitor the level of charge of the internal power supply. A minimum voltage and/or current threshold may be programmed into the controller 400, such that the controller 400 lights an LED visual alarm and/or an audible alarm to warn the user of low power condition. The alarms may be activated intermittently and at an increasing frequency as the battery approaches zero usable charge.

The controller 400 may be communicatively coupled to one or more external devices to make up a connected oxygen therapy system. The one or more external devices may be a remote external device. The one or more external devices may be an external computing device. The one or more external devices may also include sensors to collect physiological data.

FIG. 1N illustrates one implementation of a connected oxygen therapy system 450, in which the controller 400 may include the cellular wireless module (CWM) 430, or other wireless communications module, configured to allow the controller 400 to communicate, using a wireless communication protocol such as the Global System for Mobile Telephony (GSM) or other protocol (e.g., WiFi), with a remote computing device 460 such as over a network. The remote computing device 460 (or a remote external device 464) such as a cloud-based server may exchange data with the controller 400. The remote external device may be a remote computing device, such as a portable computing device. For instance, the controller 400 may also include a short range wireless module (SRWM) 440 configured to enable the controller 400 to communicate, using a short range wireless communication protocol such as Bluetooth, with a portable (mobile) computing device 466 such as a smartphone. The portable computing device 466 may be associated with a user of the POC 100 in FIG. 1A. When there are two or more external devices, each external device may be the same or different. For example, when there are two external devices and each external device is the same, there may be two servers 460. Alternatively, when there are two external devices and each external device is different, there may be the server 460 and the portable computing device 466.

The server 460 may also be in wireless communication with the portable computing device 466 using a wireless communication protocol such as GSM. A processor of the portable computing device 466 may execute an application program known as an “app” to control the interaction of the portable computing device 466 with the POC 100, the user, and/or the server 460.

The server 460 may also be in communication with a personal computing device 464 via a wired or wireless connection to a wide-area network 470 such as the Internet or Cloud, or a local-area network such as an Ethernet. A processor of the personal computing device 464 may execute a “client” program to control the interaction of the personal computing device 464 with the server 460. One example of a client program is a browser.

Further functions that may be implemented with or by the controller 400 are described in detail in other sections of this disclosure. The controller 400 may receive physiological data from the internal sensors described herein in the POC 100. Alternatively, the controller 400 may collect physiological or related data from an external blood oxygenation sensor 436 and other external sensors 438 that may be either stand-alone sensors or sensors on a health monitoring or other device. The collected physiological data may be analyzed by the server 460 or the portable computing device 466 and additional control instructions may be provided for internal registers in the controller 400.

The POC 100 collects operational data and transmits the collected operational data to a remote health data analysis engine 472 that may be executed on the server 460.

One example of operational data is usage data (when, for how long, and at what setting the POC was used). Usage data may be correlated with geolocation data indicating where the usage occurred. Geolocation data may be obtained from geolocation devices such as the GPS receiver 434 located on the POC 100 or a GPS receiver located on the portable computing device 466. Other types of operational data include output pressure and flow rate data from the pressure sensor 194 and the flow rate sensor 185.

Another example of operational data that may be collected by the POC 100 is the ratio of “auto-pulse”-delivered boluses to the total number of boluses delivered during a therapy session (see below). A larger ratio is an indication that more inhalations are going undetected and therefore that breathing is shallower/more erratic.

The health data analysis engine 472 receives the collected operational and physiological data from the POC 100 and analyzes the collected data. The health data analysis engine 472 may also receive and analyze other relevant data from external databases such as a patient information database. Other external databases may also provide additional data to the health data analysis engine. For example, a database may provide data from other POCs and corresponding patients. The database may also store relevant external data from other sources such as environmental data, scientific data, and demographic data. Data from the database and patient data may be further correlated by a machine-learning engine 480 as described below. External devices such as the personal computing device 464, accessible by a health care provider, may be connected to the health data analysis engine 472, as described below.

Physiological and other data may be collected from additional external sensors such as the external sensor 438 in FIG. 1N. In one example, the external sensor 438 may be on a body-mounted health monitoring device. Such a device may be smart wearable clothing, smart watch, or other smart devices, in order to capture data in a low impact manner continuously from the patient. For example, the health monitoring device may include one or more sensors such as an audio sensor, a heart rate sensor, a respiratory sensor such as a spirometer (to measure lung capacity), a ECG sensor, a photoplethysmography (PPG) sensor, an infrared sensor, a photoacoustic exhaled carbon dioxide sensor, an activity sensor, a radio frequency sensor, a SONAR sensor, an optical sensor, Doppler radar motion sensors, a thermometer, or impedance, piezoelectric, photoelectric, or strain gauge type sensors. This data can be fused with other data sources collected during the day or data collected during certain periods of time, such as operational data from the POC 100. The additional external sensor 438 may also be mounted on the airway delivery device such as the nasal cannula 196.

An optional internal audio sensor may be embedded in the POC 100 to detect specific patient sounds. An optional external audio sensor such as a microphone may be located on the exterior of the POC 100 to collect additional audio data. Additional sensors such as a room-temperature sensor, a contact or non-contact body temperature sensor, a room humidity sensor, a proximity sensor, a gesture sensor, a touch sensor, a gas sensor, an air quality sensor, a particulate sensor, an accelerometer, a gyroscope, a tilt sensor, acoustic sensors such as passive or active SONAR, an ultrasonic sensor, a radio frequency sensor, an accelerometer, a light intensity sensor, a LIDAR sensor, an infrared sensor (passive, transmissive, or reflective), a carbon dioxide sensor, a carbon monoxide sensor, or a chemical sensor, may be connected to the controller 400 via an external port. Data from such additional sensors may also be collected by the controller 400. Data from such additional sensors may be collected by central controller 400 on a periodic basis. Such data generally relates to the operational state of the POC 100 or its operating environment.

In one such example, the external sensor 438 may be an environmental sensor located on an air quality monitoring device connected to the controller 400. Environmental sensors 438 on an air quality monitoring device may detect gases, fumes, smoke, or particulates, in the environs of the POC 100. Such sensors may measure the quantity or density of PM2.5 (inhalable particles with a diameter of generally 2.5 micrometers and smaller) and PM10 (particles with a diameter of 10 micrometers and smaller) in the environs of the POC 100.

Data from the additional sensors may be sent to the mobile computing device 466 that may be in communication with the POC 100. Alternatively, data from the additional external sensors 438 may be directly sent to the POC 100. Data from the external sensors 438 on the health monitoring device, the POC 100, the air quality monitoring device, or the mobile computing device 466 may be transmitted to the network 470.

According to the present technology, the POC 100 may include electronic components to act as a communications hub to manage data transfer with other sensors in the vicinity of the patient, and transfer of the collected data for remote processing by the health data analysis engine 472. Such data may be collected from external sensors such as the external sensor 438 on the health monitoring device by the POC 100 even when the POC 100 is not actively delivering oxygen. Alternatively, the mobile computing device 466 may collect data from the external sensor 438, the POC 100, and other data sources, and thus serve as a communications hub to manage data transfer to the health data analysis engine 472. Other devices such as home digital assistants that may communicate with the POC 100 may also serve as the communications hub.

The control panel 600 serves as an interface between a user and the controller 400 to allow the user to initiate predetermined operation modes of the oxygen concentrator 100 and to monitor the status of the system. FIG. 1O depicts an implementation of the control panel 600. Charging input port 605, for charging the internal power supply 180, may be disposed in control panel 600.

In some implementations, the control panel 600 may include buttons to activate various operation modes for the oxygen concentrator 100. For example, the control panel 600 may include a power button 610, flow rate setting buttons 620 to 626, an active mode button 630, a sleep mode button 635, an altitude button 640, and a battery check button 650. In some implementations, one or more of the buttons may have a respective LED that may illuminate when the respective button is pressed, and may power off when the respective button is pressed again. The power button 610 may power the system on or off. If the power button 610 is activated to turn the system off, the controller 400 may initiate a shutdown sequence to place the system in a shutdown state (e.g., a state in which both canisters are pressurized). The flow rate setting buttons 620, 622, 624, and 626 allow the prescribed continuous flow rate of oxygen enriched air to be selected (e.g., 0.2 LPM by the button 620, 0.4 LPM by the button 622, 0.6 LPM by the button 624, and 0.8 LPM by the button 626). In other implementations, the number of flow rate settings may be increased or decreased. After a flow rate setting is selected, oxygen concentrator 100 will then control operations to achieve production of the oxygen enriched air according to the selected flow rate setting. The altitude button 640 may be activated when a user is going to be in a location at a higher elevation than the oxygen concentrator 100 is regularly used by the user.

The battery check button 650 initiates a battery check routine in the oxygen concentrator 100 which results in a relative battery power remaining LED 655 being illuminated on the control panel 600.

A user may have a low breathing rate or depth if relatively inactive (e.g., asleep, sitting, etc.) as estimated by comparing the detected breathing rate or depth to a threshold. The user may have a high breathing rate or depth if relatively active (e.g., walking, exercising, etc.). An active/sleep mode may be estimated automatically from breathing rate or depth, and/or the user may manually indicate active mode or sleep mode by pressing the button 630 for active mode or the button 635 for sleep mode.

The methods of operating and monitoring the POC 100 described below may be executed by the one or more processors, such as the one or more processors 410 of the controller 400, configured by program instructions, such as including, as previously described, the one or more functions and/or associated data corresponding thereto, stored in a memory such as the memory 420 of the POC 100. Alternatively, some or all of the steps of the described methods may be similarly executed by one or more processors of an external computing device, such as the server 460, forming part of the connected oxygen therapy system 450, as described above. In this latter implementation, the processors 410 may be configured by program instructions stored in the memory 420 of the POC 100 to transmit to the external computing device the measurements and parameters necessary for the performance of those steps that are to be carried out at the external computing device.

The main use of the oxygen concentrator 100 is to provide supplemental oxygen to a user. One or more flow rate settings may be selected on a control panel 600 of the oxygen concentrator 100, which then will control operations to achieve production of the oxygen enriched air according to the selected flow rate setting. In some versions, a plurality of flow rate settings may be implemented (e.g., six flow rate settings). As described in more detail herein, the controller 400 may implement a POD (pulsed oxygen delivery) or demand mode of operation. Controller 400 may regulate the size of the one or more released pulses or boluses to achieve delivery of the oxygen enriched air according to the selected flow rate setting.

In order to maximize the effect of the delivered oxygen enriched air, the controller 400 may be programmed to synchronize the release of each bolus of the oxygen enriched air with the user's inhalations. Releasing a bolus of oxygen enriched air to the user as the user inhales may prevent wastage of oxygen by not releasing oxygen, for example, when the user is exhaling. The flow rate settings on the control panel 600 may correspond to minute volumes (bolus volume multiplied by breathing rate per minute) of delivered oxygen, e.g., 0.2 LPM, 0.4 LPM, 0.6 LPM, 0.8 LPM, 1 LPM, 1.1 LPM.

Oxygen enriched air produced by oxygen concentrator 100 is stored in the oxygen accumulator 106 and, in a POD mode of operation, released to the user as the user inhales. The amount of oxygen enriched air provided by the oxygen concentrator 100 is controlled, in part, by the supply valve 160. In an implementation, the supply valve 160 is opened for a sufficient amount of time to provide the appropriate amount of oxygen enriched air, as estimated by the controller 400, to the user. In order to minimize the wastage of oxygen, the oxygen enriched air may be provided as a bolus soon after the onset of a user's inhalation is detected. For example, the bolus of oxygen enriched air may be provided in the first few milliseconds of a user's inhalation.

In an implementation, a sensor such as a pressure sensor 194 may be used to determine the onset of inhalation by the user. For example, the user's inhalation may be detected by using the pressure sensor 194. In use, the delivery conduit 192 for providing oxygen enriched air is coupled to the user's nose and/or mouth through the nasal airway delivery device 196 and/or the mouthpiece 198. The pressure in the delivery conduit 192 is therefore representative of the user's airway pressure and hence indicative of user respiration. At the onset of inhalation, the user begins to draw air into their body through the nose and/or mouth. As the air is drawn in, a negative pressure is generated at the end of the delivery conduit 192, due, in part, to the Venturi action of the air being drawn across the end of the delivery conduit 192. Controller 400 analyzes the pressure signal from the pressure sensor 194 to detect a drop in pressure indicating the onset of inhalation. Upon detection of the onset of inhalation, the supply valve 160 is opened to release a bolus of oxygen enriched air from the accumulator 106.

A positive change or rise in the pressure in delivery conduit 192 indicates an exhalation by the user. The controller 400 may analyze the pressure signal from the pressure sensor 194 to detect a rise in pressure indicating the onset of exhalation. In one implementation, when a positive pressure change is sensed, the supply valve 160 is closed until the next onset of inhalation is detected. Alternatively, the supply valve 160 may be closed after a predetermined interval known as the bolus duration.

By measuring the intervals between adjacent onsets of inhalation, the user's breathing rate may be estimated. By measuring the intervals between onsets of inhalation and the subsequent onsets of exhalation, the user's inspiratory time may be estimated.

In other implementations, the pressure sensor 194 may be located in a sensing conduit that is in pneumatic communication with the user's airway, but separate from the delivery conduit 192. In such implementations the pressure signal from the pressure sensor 194 is therefore also representative of the user's airway pressure.

In some implementations, the sensitivity of the pressure sensor 194 may be affected by the physical distance of the pressure sensor 194 from the user, especially if the pressure sensor 194 is located in oxygen concentrator 100 and the pressure difference is detected through delivery conduit 192 coupling the oxygen concentrator 100 to the user. In some implementations, the pressure sensor 194 may be placed in the airway delivery device 196 used to provide the oxygen enriched air to the user. A signal from the pressure sensor 194 may be provided to the controller 400 in the oxygen concentrator 100 electronically via a wire or through telemetry such as through Bluetooth™ or other wireless technology.

In some implementations, if the POC 100 is in active mode and an onset of inhalation has not been detected for a predetermined interval, e.g. 8 seconds, the POC 100 changes to sleep mode. Then, if onset of inhalation is not detected for a further predetermined interval (e.g. 8 seconds), the POC 100 enters “auto-pulse” mode. In auto-pulse mode, the controller 400 controls actuation of the supply valve 160 so as to deliver boluses at regular, predetermined intervals, e.g. 4 seconds. The POC 100 exits auto-pulse mode once onset of inhalation is detected by the triggering process or the POC 100 is powered off.

In some implementations, if the user's current activity level, such as that estimated using the detected user's breathing rate, exceeds a predetermined threshold, the controller 400 may implement an alarm (e.g., visual and/or audible) to warn the user that the current breathing rate is exceeding the delivery capacity of the oxygen concentrator 100. For example, the threshold may be set at 40 breaths per minute (BPM).

As described above, a device such as the sensor 436 in FIG. 1N may be used to collect blood oxygenation data from the user of the POC 100. For example, the blood oxygenation sensor 436 may be a body-worn blood oxygenation monitor such as a wrist monitor or a waist mounted monitor. Such a monitor measures blood oxygenation by photoplethysmography. The body-worn monitor may include a transmitter that communicates with an external device such as the POC 100 (through the CWM 430) or the portable computing device 466.

Another alternative external blood oxygenation sensor may be a finger-clip device that measures blood oxygenation levels. Such a device may also measure blood oxygenation by photoplethysmography. In this example, the finger-clip device such as the Onyx, WristOx2, or NoninConnect devices manufactured by Nonin may be in wireless communication with an external device. Another example may be an ear mounted device such as the BCI 3301 hand held pulse oximeter with 3078 ear sensor manufactured by Turner Medical. Alternatively, the user or a health care professional may take the blood oxygenation measurement and enter the measurement into the app running on the portable computing device 466.

Another alternative is that the blood oxygenation sensor 436 may be installed in the POC 100. Such a sensor may be a finger-clip device with a physical aperture in the housing of the POC 100 for a user to insert their finger. The sensor communicates the measured blood oxygenation data directly to the controller 400. Other implementations may include adapting existing body worn sensors such as a wrist-worn device such as a Fitbit, the Loop by Spry Health, the BORAband by Biosency, or an Apple watch. The sensor 436 may be an implanted sensor that communicates data through a wireless receiver. Examples of an implanted sensor may include a skin-applied patch or a one-time nanobot implant.

In another alternative, the POC 100 itself may be a wearable device, and the blood oxygenation sensor 436 may be installed in the POC 100 so as to be in contact with the user's skin when worn. The external sensors 438 may in this implementation be mounted on the POC 100 rather than on the health monitoring device.

In the connected oxygen therapy system 450, the example POC 100 serves as a connected hub in the home environment. The POC 100 in its hub role cooperates with the external health data analysis engine 472 for managing health conditions such as COPD, asthma, emphysema, and chronic bronchitis in this example. This management could be offered as a service to an integrated payor. The POC 100 and associated external sensors 438, such as those mounted on a health monitoring device, can monitor many aspects of respiratory conditions that the patient may be suffering from.

The collected data may be used to determine respiration changes for the tracking of changes in health conditions (e.g., higher than normal breathing rate/tachypnea). The collection of respiration data over time may determine how base breathing rate evolves over time. Such disease analysis may include tracking worsening health conditions. For example, the collected data may be analyzed to detect worsening asthma, pollen allergy, common cold, or respiratory tract infections.

Audio data may be used to confirm or enhance health data analysis. For example, sounds of breathing from a patient from an internal audio sensor may be used in conjunction with external sounds detected by an external audio sensor to determine audio data.

The audio data may include the level of residual snoring, gasping, coughing, wheezing, spluttering, and the sound of the heartbeat. These sounds may be used to monitor respiratory disease and other health conditions. For example, the intensity and timing (inspiration or expiration) of a wheeze sound may be a symptom of respiratory conditions, disorders, or ailments. In addition, lack of sounds, such as a silent chest, may indicate severe asthma in combination with other vital signs like a higher heart rate and breathing rate.

The collected operational and physiological data may be analyzed in the context of patient-specific conditions that may be derived from data from other sources. Such data may include outcomes reported by the patient through an interface generated by the app running on the mobile computing device 466, or input from electronic health records on a database. The patient-specific data may therefore include co-morbidities, demographic details (body mass index (BMI), age, gender), geographic details (allergen risks due to pollen count, heat exhaustion due to outside temperature, air quality and oxygen levels due to altitude), and medications associated with the patient. The patient-reported outcomes (PROs) may include subjective feedback on how the patient is feeling (wellbeing), whether the patient feels fatigued, and the patient's level of sleepiness.

A sensor on the POC 100 may sense gas (breath) from the patient using for example an array of cross-reactive sensors, and pattern recognition/deep learning to identify the characteristic changes in certain volatile organic compounds (VOCs) due to disease progression.

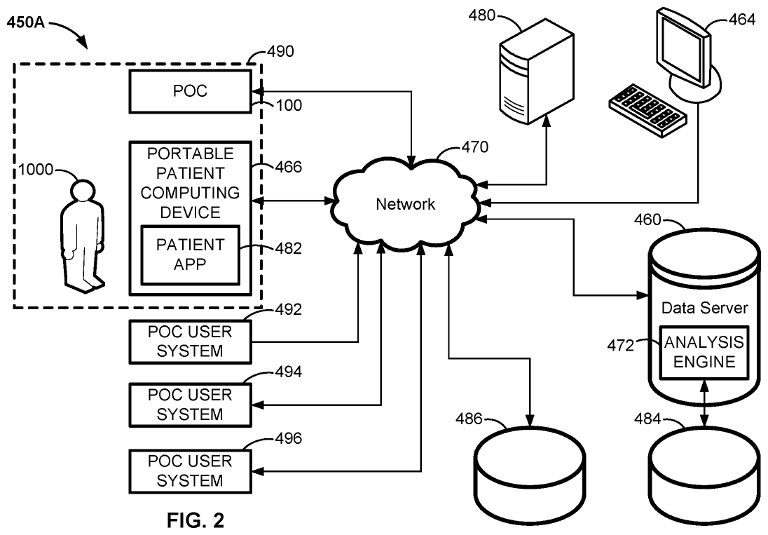

FIG. 2 illustrates another implementation of a connected oxygen therapy system 450A, in which the controller 400 of the POC 100 includes the CWM 430 as in FIG. 1N configured to allow the controller 400 to communicate, using a wireless communication protocol such as the Global System for Mobile Telephony (GSM) or other protocol (e.g., WiFi), with a remote external device (or remote computing device) such as the cloud-based server 460 over the network 470. The network 470 may be a wide-area network such as the Internet or cloud, or a local-area network such as an Ethernet. Alternatively, or in addition, the remote external device may be a remote computing device, such as the portable computing device 466. For instance, the controller 400 may also include the short range wireless module 440 configured to enable the controller 400 to communicate, using a short range wireless communication protocol such as Bluetooth™, with the portable computing device 466 such as a smartphone. The portable computing device 466 may be associated with a user 1000 of the POC 100.

The server 460 may also be in wireless communication with the portable computing device 466 using a wireless communication protocol such as GSM. A processor of the portable computing device 466 may execute a patient engagement program or “app” 482 to control the interaction of the smartphone with the POC 100, the user 1000, and/or the server 460. The patient engagement app 482 may include input interfaces for the user 1000 to input data such as SpO2 readings from external sensors if a networked blood oxygenation sensor such as the sensor 436 in FIG. 1N is not available.

The server 460 includes an analysis engine 472 that may execute operations such as analysing received data to determine and respond to adverse environmental conditions. The server 460 may also be in communication with other devices such as a personal computing device 464 via a wired or wireless connection via the network 470. The server 460 has access to a database 484 that stores operational and physiological data about the POCs and users managed by the connected oxygen therapy system 450. The database 484 may be segmented into individual databases such as a user database having physiological data about users of the POCs and operational data associated with the POC use. The server 460 may also be in communication via the network 470 with other relevant databases such as an environmental database 486 that may provide additional data.

The user 1000 of the POC 100 and portable computing device 466 may be organized as a POC user system 490. The connected oxygen therapy system 450A may comprise multiple POC user systems 490, 492, 494 and 496 that each include a POC user, POCs such as the POC 100, and portable computing devices such as the portable computing device 466. Each of the other POC user systems 492, 494 and 496 are in communication with the server 460, either directly or via respective portable computing devices associated with respective users of the POCs. Controllers corresponding to the controller 400 and transceivers corresponding to the CWM 430 in each of the POCs of the systems 492, 494 and 496 collect and transmit the data described above in relation to FIG. 1N. The personal computing device 464 may be associated with a health management entity (HME) that is responsible for the therapy of a population of users of the fleet of POCs.

Data from the database 484, analysis results from the health data analysis engine 472 and data from individual POC user systems such as the user system 490 may be further correlated by the machine-learning engine 480. The machine-learning engine 480 may implement machine-learning structures such as a neural network, decision tree ensemble, support vector machine, Bayesian network, or gradient boosting machine. Such structures can be configured to implement either linear or non-linear predictive models for monitoring different health conditions. For example, data processing such as determining the status of a patient's health condition may be carried out by any one or more of supervised machine learning, deep learning, a convolutional neural network, and a recurrent neural network. In addition to descriptive and predictive supervised machine learning with hand-crafted features, it is possible to implement deep learning on the machine-learning engine 480. This typically relies on a larger amount of scored (labeled) data (such as many hundreds of data points from different POC devices) for normal and abnormal conditions. This approach may implement many interconnected layers of neurons to form a neural network (“deeper” than a simple neural network), such that more and more complex features are “learned” by each layer. Machine learning can use many more variables than hand-crafted features or simple decision trees.

Convolutional neural networks (CNNs) are used widely in audio and image processing for inferring information (such as for face recognition), and can also be applied to audio spectrograms, or even population scale genomic data sets created from the collected data represented as images. When carrying out image or spectrogram processing, the system cognitively “learns” temporal and frequency properties from intensity, spectral, and statistical estimates of the digitized image or spectrogram data.

In contrast to CNNs, not all problems can be represented with fixed-length inputs and outputs. For example, processing respiratory sounds or sounds of the heart has similarities with speech recognition and time series prediction. Thus, the sound analysis can benefit from a system to store and use context information such as recurrent neural networks (RNNs) that can take the previous output or hidden states as inputs. In other words, they may be multilayered neural networks that can store information in context nodes. RNNs allow for processing of variable length inputs and outputs by maintaining state information across time steps, and may include LSTMs (long short term memories, types of “neurons” to enable RNNs increased control over, which can be unidirectional or bidirectional) to manage the vanishing gradient problem and/or by using gradient clipping.

The machine-learning engine 480 may be trained for supervised learning of known patient status from known data inputs for assistance in analyzing input data. The machine-learning engine 480 may also be trained for unsupervised learning to determine unknown correlations between input data and patient status, to increase the range of analysis of the health data analysis engine 472.

The collection of data from the population of users of the fleet of POC user systems such as user systems 492, 494 and 496 allows a large data set to be built up for the purpose of providing more accurate health data analysis. As described above, the collected data may be supplied to the machine-learning engine 480 for further analysis. The analysis from the health data analysis engine 472 and/or the machine-learning engine 480 may be used to provide health data analysis for any of the individual users such as the user 1000.

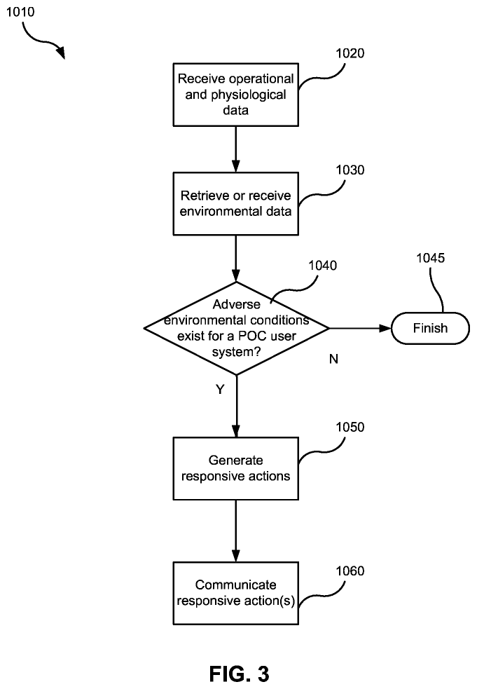

FIG. 3 is a flow chart illustrating a method 1010 for analyzing and responding to local environmental conditions, as implemented in a connected oxygen therapy system such as the system 450A of FIG. 2. The method 1010 may be executed by the analysis engine 472 running on the server 460. The method 1010 starts at step 1020, which receives the operational and physiological data transmitted by POC user systems in the connected oxygen therapy system, system, such as the POC user system 490. As mentioned above, examples of physiological data are breathing rate and inspiratory time. As mentioned above, examples of operational data are usage data and geolocation data.

Step 1030 then receives environmental data transmitted by POC user systems such as the POC user system 490. Alternatively, step 1020 may retrieve environmental data that is local to a particular POC user system, e.g., the POC user system 490, from an environmental database 486 based on the geolocation data received from the POC user system at step 1020. As mentioned above, an example of environmental data is air quality, as measured by quantity or density of particulates such as PM2.5 and PM10, in the environs of the POC user system 490. The data collected at steps 1020 and 1030 is stored in a database 484.

The next step 1040 analyzes the environmental, physiological, and operational data collected at steps 1020 and 1030 to determine whether adverse environmental conditions exist local to a particular POC user system 490. For example, step 1040 may compare an air quality measure, such as a density of particulates, local to each POC user system with a threshold of poor air quality. If adverse environmental conditions do not exist (“N”), the method 1010 finishes at step 1045. If adverse environmental conditions do exist for a particular POC user system (“Y”), e.g., the POC user system 490, the method 1010 proceeds to step 1050. Step 1050 conducts further analysis of the data to determine a responsive action for the POC user system 490 to lessen the effects of the adverse environmental conditions. One example of a responsive action is to control the POC 100 such as increasing the flow rate setting in use at the current therapy session. The controller 400 of the POC 100 may receive such a command to increase the volume of the bolus or the frequency of oxygen delivery. As such, the responsive action may control the POC 100 by changing the flow of oxygen to the user via the controller 400 which is configured to control the POC 100. Consequently, the responsive action may change flow control of oxygen to the user. Thus, the responsive action is controlling the POC 100 based on the collected data. Another example is to provide a recommendation that the user 1000 not go out of doors. Yet another example is providing a recommendation that the user 1000 take the POC 100 with them if they do go out of doors. Another example is to provide a recommendation to replace the particulate filter 187.

The next step 1060 then communicates the responsive action to the user 1000 of the POC user system 490 via the network 470. In some implementations, the communication is made via a “push” notification on an interface generated by the patient engagement app 482. In other implementations, the communication is made via the display on the control panel 600 of the POC 100. One example of such a communication is a displayed message such as “poor air quality today, please use setting 4”.

Step 1050 may be carried out with the assistance of the machine-learning engine 480. Over time, the database 484 builds up a large data set of previous instances of adverse environmental conditions and their effects on users' physiology under various usage scenarios such as usage at different flow rate settings at different levels of indoor and outdoor activity. From this, the machine-learning engine 480 may learn correlations between usage scenarios, adverse environmental conditions, and positive outcomes such as an absence of dyspnea or desaturation events in groups of similar users, using the machine learning techniques described above. Once such correlations are learned, the analysis engine 472 to implement step 1050 may determine a group of users that is similar to the user 1000 of the POC user system 490 determined at step 1040. The analysis engine 472 then determines a usage scenario that is correlated with a positive outcome for users in that group under similar adverse environmental conditions to those which triggered the “Y” at step 1040. The responsive action generated at step 1050 is the action which brings about the usage scenario correlated with a positive outcome for the group of users. The data collected relating to location may also be used to refine treatment for respiratory ailments such as by adjusting treatments to account for environmental factors.

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

While various embodiments of the present invention have been described above, it should be understood that they have been presented by way of example only, and not limitation. Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application. Thus, the breadth and scope of the present invention should not be limited by any of the above described embodiments. Rather, the scope of the invention should be defined in accordance with the following claims and their equivalents.

### LABEL LIST

| | |
|---|---|
| oxygen concentrator | 100 |
| inlet | 101 |
| inlet | 105 |
| accumulator | 106 |
| accumulator pressure sensor | 107 |
| inlet muffler | 108 |
| inlet valve | 122 |
| valve | 124 |
| filter | 129 |
| outlet | 130 |
| outlet valve | 132 |
| muffler | 133 |
| outlet valve | 134 |
| spring baffle | 139 |
| check valve | 142 |
| check valve | 144 |
| flow restrictor | 151 |
| valve | 152 |
| flow restrictor | 153 |
| valve | 154 |
| flow restrictor | 155 |
| supply valve | 160 |
| expansion chamber | 162 |
| ultrasonic sensor | 165 |
| emitter | 166 |
| receiver | 168 |
| housing | 170 |
| fan | 172 |
| outlet | 173 |
| outlet port | 174 |
| flow restrictor | 175 |
| power supply | 180 |
| flow rate sensor | 185 |
| filter | 187 |
| connector | 190 |
| delivery conduit | 192 |
| pressure sensor | 194 |
| nasal cannula airway delivery device | 196 |
| mouthpiece | 198 |
| compression system | 200 |
| speed sensor | 201 |
| compressor | 210 |
| compressor outlet | 212 |
| motor | 220 |
| external rotating armature | 230 |
| air transfer device | 240 |
| compressor outlet conduit | 250 |
| canister system | 300 |
| canister | 302 |
| canister | 304 |
| air inlet | 306 |
| housing component | 310 |
| base | 315 |
| valve seat | 322 |
| opening | 323 |
| valve seat | 324 |
| outlet | 325 |
| exhaust gas | 327 |
| inlet conduit | 330 |
| valve seat | 332 |
| aperture | 337 |
| conduit | 342 |
| conduit | 344 |
| conduit | 346 |
| opening | 375 |
| controller | 400 |
| processor | 410 |
| memory | 420 |
| cellular wireless module | 430 |
| GPS receiver | 434 |
| sensor | 436 |
| external sensor | 438 |
| short range wireless module | 440 |
| oxygen therapy system | 450 |
| oxygen therapy system | 450A |
| server | 460 |
| personal computing device | 464 |
| mobile computing device | 466 |
| network | 470 |
| health data analysis engine | 472 |
| machine - learning engine | 480 |
| patient engagement app | 482 |
| database | 484 |
| environmental database | 486 |
| user system | 490 |
| user system | 492 |
| user system | 494 |
| housing component | 510 |
| conduit | 530 |
| conduit | 532 |
| conduit | 534 |
| opening | 542 |
| opening | 544 |
| valve seat | 552 |
| valve seat | 554 |
| control panel | 600 |
| input port | 605 |
| power button | 610 |
| flow rate setting button | 620 |
| flow rate setting button | 622 |
| button | 624 |
| button | 626 |
| button | 630 |
| button | 635 |
| altitude button | 640 |
| battery check button | 650 |
| LED | 655 |
| user | 1000 |
| method | 1010 |
| step | 1020 |
| step | 1030 |
| step | 1040 |
| step | 1045 |
| step | 1050 |
| step | 1060 |

What is claimed is:

1. A method of responding to adverse environmental conditions local to a user of an oxygen concentrator, the method comprising:
collecting physiological data of the user;
collecting operational data of the oxygen concentrator during operation of the oxygen concentrator;
collecting environment data local to the oxygen concentrator;
determining, based on the collected environmental data, whether adverse environmental conditions exist local to the oxygen concentrator;
analysing the collected physiological, operational, and environmental data to determine a responsive action to the determined adverse environmental conditions; and
communicating the responsive action to the user.

2. The method of claim 1, wherein the operational data comprises geolocation data.

3. The method of claim 2, wherein collecting the environmental data comprises retrieving environmental data from an environmental database using the geolocation data.

4. The method of claim 1, wherein the environmental data comprises an air quality measure.

5. The method of claim 4, wherein determining whether adverse environmental conditions exist comprises comparing the air quality measure to a threshold representative of poor air quality.

6. The method of claim 1, wherein the analysing uses physiological, operational, and environmental data collected from other portable oxygen concentrators stored in a database.

7. The method of claim 6, further comprising storing the collected physiological, operational, and environmental data in the database.

8. The method of claim 1, wherein the responsive action is controlling the oxygen concentrator to change flow control of oxygen to the user.

9. A connected oxygen therapy system comprising:
an oxygen concentrator configured to generate oxygen enriched air for delivery to a user;
a physiological sensor configured to collect physiological data of the user;
an operational sensor configured to collect operational data of the oxygen concentrator during operation of the oxygen concentrator;
a processor configured to:
collect physiological data of the user;
collect operational data of the oxygen concentrator during operation of the oxygen concentrator;
collect environment data local to the oxygen concentrator is collected;
based on the collected environmental data, determine whether adverse environmental conditions exist local to the oxygen concentrator;
analyzing the collected physiological, operational, and environmental data to determine a responsive action to the determined adverse environmental conditions; and
communicating the responsive action to the user.

10. The connected oxygen therapy system of claim 9, further comprising a server in communication with the oxygen concentrator, wherein the processor is a processor of the server.

11. The system of claim 10, further comprising a portable computing device configured to act as an intermediary between the oxygen concentrator and the server.

12. The system of claim 1, further comprising an environmental sensor configured to generate the environmental data local to the oxygen concentrator.

13. The system of claim 1, further comprising a geolocation device configured to generate geolocation data of the oxygen concentrator.

14. The system of claim 13, further comprising an environmental database comprising environmental data.

15. The system of claim 14, wherein the processor is configured to retrieve the environmental data from the environmental database using the geolocation data.

16. The system of claim 1, further comprising a database on which physiological, operational, and environmental data from other oxygen concentrators are stored.

17. The system of claim 16, wherein the processor is configured to use the physiological, operational, and environmental data from other oxygen concentrators in the analysing.

18. The system of claim 1, wherein the responsive action is controlling the oxygen concentrator to change flow control of oxygen to the user, and wherein the processor is configured to control the oxygen concentrator.
